(19) United States
(12) Patent Application Publication
(10) Pub. No.: US 2022/0339116 A1
(43) Pub. Date: Oct. 27, 2022

(54) LIPID NANOPARTICLES AS ORAL VEHICLES OF IMMUNOTHERAPY

(71) Applicant: The Johns Hopkins University, Baltimore, MD (US)

(72) Inventors: Giorgio Raimondi, Severna Park, MD (US); Julia Patrone, Ellicott City, MD (US); Xiomara Calderon-Colon, Fulton, MD (US); Olivia Tiburzi, Silver Spring, MD (US)

(21) Appl. No.: 17/600,843

(22) PCT Filed: Apr. 3, 2020
(86) PCT No.: PCT/US2020/026574

§ 371 Date: Oct. 1, 2021

Related U.S. Application Data
(60) Provisional application No. 62/858,775, filed on Jun. 7, 2019.
(60) Provisional application No. 62/828,743, filed on Apr. 3, 2019.

(51) Int. Cl. A61K 9/51; A61K 31/519; A61K 31/541; A61K 38/28

(52) U.S. Cl. CPC A61K 9/5123; A61K 31/519; A61K 31/541; A61K 38/28

(57) ABSTRACT

Lipid nanoparticle compositions for use in immunomodulation and treatment of autoimmune and related diseases such as Type 1 Diabetes are provided along with methods for using same.

### REFERENCE TO RELATED APPLICATIONS

This application is a § 371 U.S. National Entry Application of PCT/US2020/026574, filed Apr. 3, 2020, which claims the benefit of U.S. Provisional Patent Application No. 62/828,743, filed on Apr. 3, 2019 and U.S. Provisional Patent Application No. 62/858,775, filed on Jun. 7, 2019, each of which are hereby incorporated by reference for all purposes as if fully set forth herein.

### STATEMENT OF GOVERNMENTAL INTEREST

This invention was made with government support under grant HL127355 awarded by the National Institutes of Health. The government has certain rights in the invention.

### BACKGROUND OF THE INVENTION

Autoimmune diseases are pathologies caused by the erroneous attack of a patient immune system against an organ or tissues of the own body. Autoimmune diseases are often treated with immunosuppressants that weaken the entire immune system, leaving patients vulnerable to infection. An ideal autoimmune therapy establishes immune tolerance to the specific self-antigen(s) recognized by the host immune system, while the rest of the immune system remains intact. A prototypical example is Type-1 diabetes (T1D), an autoimmune disease caused by the destruction of pancreatic β cells by autoreactive T cells. Unfortunately, there is still no successful cure for T1D and diabetic individuals are dependent on lifelong, continuous insulin administration. Despite the use of various technologies for insulin delivery, many patients with T1D suffer devastating consequences associated with poor glycemic control including accelerated cardiovascular and perivascular diseases, nephropathy, retinopathy, neuropathy, oral diseases and premature death. Moreover, the incidence of T1D appears to be increasing worldwide, which clearly justifies the intense investigation of treatments that would prevent the development of this disease in people at risk, or stabilize (and even improve) the function of p cells in patients with recent T1D onset. Unfortunately, most of the clinical trials conducted in the past decade (focused on immune intervention) have failed to successfully preserve p cell function, or have shown only a transient effect.

There is a great opportunity to learn from all these previous clinical trials and identify what can be applied to future studies. There is a consensus that new therapies designed to minimize treatment interventions, while achieving antigen-specific autoimmune tolerance, would be transformative for this disease. An ideal therapeutic strategy would be non-invasive, physiologically targeted, and would involve a limited treatment regimen to achieve pancreatic 3 cell-specific immune tolerance. Appreciating that multiple immune pathways contribute to the etiology of T1D, a consensus is also growing toward the need for a “combination therapeutic approach.” This would include: 1) an anti-inflammatory agent that would control the release of inflammatory cytokines like IL-1 and TNF-α; 2) an immunomodulatory agent that would promote the control of T cells and B cells and favor the activity and accumulation of regulatory T cells; 3) a source of diabetes-related antigen(s) to tailor the immune-intervention in an antigen-specific fashion.¹

Multiple reports have indicated in animal models of diabetes^{2,3} as well as in diabetic patients,⁴ that regulatory T cells (Treg; a subpopulation of T lymphocytes responsible for the control of exuberant reactivity of the adaptive immune system and for the prevention of autoimmune diseases in healthy individuals) exhibit reduced regulatory efficacy. Depending on the model investigated, this defect in regulation is attributed to an intrinsic reduced suppressive activity of Treg or to a reduced susceptibility to regulation in conventional T cells (Tconv).^{5,6} Although these results could indicate that both of these “deregulations” could play in concert, they all point toward the loss of regulation of autoreactive T lymphocytes as a key process of diabetes development. Multiple evidences demonstrate that increasing the frequency of Treg (through adoptive transfer of ex vivo-expanded Treg or in vivo interventions) has beneficial effects against the development of diabetes.^{7,8} This therapeutic effect is even more pronounced when antigen-specific Treg are infused.⁹⁻¹¹ However, the beneficial effect reported is inefficient and very often transitory, with normoglycemia reverting to diabetes in a matter of weeks to months. Although these approaches correctly aim at shifting the balance from autoreactive T cells to Treg, they do not address the role of environmental components (e.g. inflammation) that contributed to the unbalance initially and can therefore cause it again. Likely, this underlies the unsatisfactory results of therapeutic clinical trials in humans.¹²⁻¹⁵ Therefore, the need for a combination therapy is apparent.

Additional strategies to attempt to restore regulation of diabetogenic T cells have derived from our understanding of T cell activation—and from the field of transplant immunology. T lymphocytes need to integrate signals delivered via both the T-cell receptor (TCR) and costimulatory receptors to orchestrate the nuclear translocation of the transcription factors necessary for activation. This fundamental function of costimulatory pathways underlies the rationale for the creation of “costimulation blockade” (CoB) therapies to control T cell activation and promote tolerance induction.¹⁷ Blockade of the CD28 pathway with CTLA4-Ig (a recombinant version of the co-regulatory molecule CTLA4 that sequesters the CD28 ligands CD80 and CD86; abatacept) proved to be a potent modulator of transplant rejection and an important component of tolerance regimens (involving, however, at least one additional biologic) in rodents.¹⁸⁻²² Prolonged administration of CTLA4-Ig in NOD mice, a widely used model of human T1D that spontaneously develop diabetes,^{13, 23, 24} was effective in preventing diabetes onset when initiated very early (at 2-3 weeks of age).²⁵ In almost all these models, the protective effect of CoB relied on promoting an anergic state in reactive T cells, while also stimulating the accumulation of Treg. However, CD28 pathway blockade with CTLA4-Ig alone does not prevent all T cell proliferation or transplant rejection, and is not sufficient to promote tolerance.^{26, 27, 28} These results can also provide the basis for understanding the limited success of the use of abatacept in a recent trial in T1D (Abatacept TrialNet).^{29, 30}

### SUMMARY OF THE INVENTION

A growing body of evidence shows that induction of long term transplant survival by CoB is impaired by inflammatory responses.³¹ It then stands to reason that in inflammatory conditions the activation of T cells can happen in a CD28 independent manner. The role of inflammatory cytokines as supporters of T cell activation has been reported.³²⁻³⁶ Recent reports on the powerful protective effect that CoB has in strain combinations where both donor and recipient were knockout for Myd88³⁷⁻³⁹ or for IL-6 and TNFα⁴⁰ support a major role of inflammatory cytokines in complementing costimulation. All these observations underscore our need for a therapeutic intervention that would preserve the immunomodulatory effects of CoB (anergy induction, deletion of alloreactive T cells, and promotion of regulatory T cells activity) by controlling the inflamed environment that otherwise negates CoB's tolerogenic potential.

In accordance with an embodiment, the present invention provides novel nanoparticle based antigen specific immunotherapies.

Differently from transplantation, where the timing of exposure to antigens is well defined, the development of autoreactivity in diseases like T1D spans a much longer period and remains quite elusive. For this reason, the present inventors show that immunotherapeutic methods for preventing or halting T1D are autoantigen-specific. These methods involve the combined administration of autoantigen(s) with a tolerance promoting conditioning of the antigen presenting cells that would then present the antigen in an effective tolerogenic fashion. There are several well-characterized auto-antigens associated with T1D including insulin, glutamic acid decarboxylase 65 (GADA), islet-antigen-2 (IA-2A), and zinc transporter 8 (ZnT8A). Autoantibodies specific for insulin are detectable at the earliest stages of disease and are present in almost all prediabetic individuals.⁴¹ Several studies have explored the administration of insulin or insulin peptides within antigen-specific immunotherapy strategies to prevent the onset of T1D.⁴²⁻⁴⁵ While subcutaneous administration of low dose insulin did not significantly delay T1D within a recent clinical trial⁴⁶, several mouse studies (using higher concentrations of insulin) have demonstrated that oral or nasal insulin administration was effective at delaying disease onset.^{47, 48}

Oral drug administration is particularly attractive as a pain free, non-invasive delivery method that favors patient compliance, and as such, would be ideal for delivery of an antigen specific therapy. Attempts to successfully deliver insulin orally have been made for decades⁴⁹. Unfortunately, administration of native insulin by this route leads to rapid enzymatic degradation in the stomach and intestinal lumen, resulting in low bioavailability.⁵⁰ Oral administration of insulin is also hampered by its low permeability with respect to the intestinal mucosa.⁵¹ The use of permeation enhancers is one strategy to improve bioavailability, but brings the risk of carrying toxins and pathogens to the systemic bloodstream with the potential for local and systemic pathophysiologic effects⁴⁹.

To achieve the goal of oral administration of an antigen specific immunotherapy, the present inventive compositions and methods take advantage of recent advances in nanotechnology-based drug-delivery applications.⁵²⁻⁵⁴ Many current research efforts are focused on polymer-derived nanoparticles (e.g. poly(lactic-co-glycolic acid) or PLGA), however limited drug loading and the potential for drug expulsion remain a challenge for therapeutic development.^{55, 56} Moreover, while successful applications of polymeric nanoparticle for immunotherapy of autoimmune diseases (like T1D) have been demonstrated when using parenteral routes of therapeutic administration, attempts to deliver these strategies employing an oral route have all been unsuccessful.

Alternative classes of lipid-based particles have emerged as having unique properties. These lipid nanoparticles (LNp), including solid lipid nanoparticles (SLN) and nanostructured lipid carriers (NLC) are especially promising platforms for drug delivery due to their biocompatibility, stability, and capacity for controlled release of a wide range of active ingredients. They are made using physiological lipids and surfactants generally regarded as safe (GRAS) by the FDA. LNp are distinct from liposomes, a carrier system that has shown promise, but is characterized by instability and drug leakage, as well as limited targeting and scalability. An additional unique property of LNp is their ability to pass biological barriers and accumulate in the lymphatic system, if properly sized.⁵⁷⁻⁶¹ This happens even with the intra-gastric administration of LNp, supporting their use as delivery system to avoid first-pass hepatic metabolism by the liver, limit systemic toxicity, and localize therapeutics into lymphoid tissues of the gastrointestinal tract, the likely physiological sites of activation of T1D autoimmune responses.

In accordance with several embodiments, the present invention provides a new strategy of intervention based on the implementation of various LNp designed to penetrate the intestinal barrier and accumulate in pancreatic and mesenteric lymph nodes, allowing controlled and localized delivery of both an anti-inflammatory agent and a T1D nominal autoantigen (insulin b-peptide). While minimizing its side effects, the inventive compositions and methods maximize locally the synergy between the anti-inflammatory agent and an optional short course of systemic CTLA4-Ig administration. This achieves what we define as “enhanced costimulation blockade” (ECoB) favoring tolerance induction in insulin-reactive T cells and promoting Treg activity. The novel compositions and methods delineate a strategy of intervention that optimizes the immunotherapeutic effect while minimizing possible side effects, and will reduce the complications associated with poor compliance as the length of the treatment will be short (and, if needed, spaced in time).

In accordance with one or more embodiments, the present inventors now show that pharmacological inhibition of the production and signaling of a class of inflammatory cytokines, through the small molecule inhibitor Tofacitinib (Tofa),⁶³ is particularly effective in synergizing with CTLA4-Ig to effectively prevent the activation of alloreactive T cells and promoting transplant survival in a model of transplantation.

As such, in accordance with an embodiment, these inventive methods defined as “Enhanced Costimulation Blockade” (ECoB), cause the accumulation of protective regulatory T cells in target tissues while minimizing the generation of pathogenic effector T cells. These effects are achieved through a continuous, but short-term, administration of Tofa, suggesting a strategy that would be transformative for patients as it would avoid chronic immunosuppression and associated deleterious side effects.

In accordance with an embodiment, the present invention provides a lipid nanoparticle comprising one or more small molecule inhibitors of inflammatory cytokines.

In accordance with an embodiment, the present invention provides a lipid nanoparticle comprising one or more small molecule inhibitors of inflammatory cytokines and a detectable moiety.

In accordance with another embodiment, the present invention provides a lipid nanoparticle comprising one or more inhibitors of Janus kinase (JAK) inhibitors.

In accordance with another embodiment, the present invention provides a lipid nanoparticle comprising the JAK inhibitor Tofacitinib.

In accordance with a further embodiment, the present invention provides a lipid nanoparticle comprising Tofacitinib and at least one immunoactive agent.

In accordance with another embodiment, the present invention provides a lipid nanoparticle comprising a disease associated antigen.

In accordance with yet another embodiment, the present invention provides a lipid nanoparticle comprising an insulin peptide.

In accordance with an embodiment, the present invention provides a method for treating an autoimmune disorder in a subject in need thereof comprising administering to the subject an effective amount of a lipid nanoparticle comprising one or more small molecule inhibitors of inflammatory cytokines.

In accordance with another embodiment, the present invention provides a method for treating T1D in a subject in need thereof comprising administering to the subject an effective amount of a lipid nanoparticle comprising one or more small molecule inhibitors of inflammatory cytokines.

In accordance with an embodiment, the present invention provides a method for Enhanced Costimulation Blockade of an autoimmune response in a subject having an autoimmune disorder comprising administering to the subject an effective amount of a lipid nanoparticle comprising one or more small molecule inhibitors of inflammatory cytokines and a checkpoint inhibitor.

### BRIEF DESCRIPTION OF THE DRAWINGS

FIGS. 1A-1B. ECoB promotes transplant survival via accumulation of Treg and inhibition of TH1 cells. TA, Survival curves of B6 to BALB/c heart transplants subjected to the treatment indicated. CTLA4-Ig administered on days 0, 2, 4, and 6. Tofa dosed daily from POD −1 to POD9. n=4-5 for each group. CTLA4-Ig+Tofa was significantly different from CTLA4-Ig alone: P=0.02. 4 h IS indicates that donor hearts were kept ischemic for 4 h before transplantation. 1B, Profile of graft infiltrating CD4 T cells and of the production of IFN-γ by splenic T cells stimulated ex vivo with PMA/Inonmycin. Representative of 3 recipients.

FIGS. 2A-2B. Jak inhibition impacts APC maturation. 2A, Representative graphs of surface expression of maturation markers on DC left untreated or exposed overnight to LPS+/−Tofa. 2B, Supernatants from DC cultures were tested for accumulation of the indicated cytokines by ELISA. n=3.

FIG. 3. Tofa does not interfere with Treg suppression of T cells. Results of CFSE-Proliferation-Assay conducted with the addition of Treg cells (at either 2:1 or 4:1 T cell to Treg ratio) and Tofa (at 0.5 or 1 μM). Values are normalized to the extent of proliferation measured with no Treg.

FIGS. 4A-4B. Effect of Tofa-Loading on Size and Crystallinity as a Function of Lipid to Surfactant Ratio. LNp were generated using the PIT method. Particle size (4A) was measured using dynamic light scattering (DLS) and latent heat of melting (4B) was measured using differential scanning calorimetry (DSC). Formulations were prepared in duplicate. Error bars represent the standard deviation of the mean.

FIGS. 5A-5B. Tofa-LNp inhibit DC maturation and function. 5A, Representative result of maturation markers expression on DC left untreated (grey), stimulated with LPS (blue), or pre-incubated with Tofa-LNp before exposure to LPS (green). 5B, Impact of Tofa-LNp on the T cell allostimulatory capacity of DC. Representative proliferation of allogeneic T cells (CD4 and CD8) stimulated by LPS-matured DC that were unmodified (left column) or pre-conditioned with Tofa-LNp (right column). Numbers indicate percentage of proliferating cells.

FIGS. 6A-6C. In vivo distribution of LNp. 6A, Representative images of fluorescent signal from HLDI-LNp at 96 h post subcutaneous injection. 6B, Fluorescent signal from Hexamethylindodicarbocyanine Iodide labeled LNp (HLDI-LNp) at 24 h post oral gavage. Msn: mesenteric. Pnc: pancreatic. Ing: inguinal lymph nodes. Spl: spleen. C, Representative cellular uptake of HLDI-LNp 48 h post injection. Cell suspensions were generated from inguinal lymph nodes of injected mice and the HLDI-fluorescent signal detected on the indicated cell populations via flow cytometry.

FIG. 7. Effect of Solid to Liquid Lipid Blend on Particle Size (blue; left y axis) and Crystallinity (yellow; right y axis). LNp were generated using the PIT method. Particle size was measured using DLS and latent heat of melting was measured using DSC. Data represent a single measurement of the listed formulations.

FIG. 8. Control LNp as imaged using TEM.

FIG. 9. Incorporation kinetic of NLC by mouse BMDC. Representative results of the incorporation of-NLC (added at either 0.1 or 1 μg/ml) by mouse dendritic cells exposed for the time indicated. Incorporation is measured as increase of fluorescence in comparison to baseline (time 0).

FIG. 10. Internalization of NLC by mouse BMDC. The observed kinetic of NLC incorporation by BMDC (FIG. 9) could result from a true internalization of the particles by the cells or by adherence of the particles to the extracellular membrane. We performed confocal microscopy analysis of HLDI-NLC exposed BMDC (1 μg/ml particles concentration, 10 minutes incubation time) to identify the cellular localization of the nanoparticles. The results, FIG. 10, clearly show that the NLC (red) are internalized by mouse BMDC (cyan) and accumulate intracellularly.

FIG. 11. Tofa-NLC prevent antigen presenting cells maturation. t-SNE plot of the cumulative expression of 4 maturation markers (CD40, CD80, CD86, MHC-II) on the surface of mouse BMDC exposed (as indicated) to LPS after co-culture with control (cNLC) or Tofa-NLC.

FIG. 12. In addition to assessing the maturation inhibition of mouse BMDC by the internalization of Tofa-NLC, we determined via Luminex technology changes in the accumulation of pro-inflammatory cytokines by maturing cells in the same treatment groups as FIG. 11. Two time points post addition of the stimulus (LPS) were analyzed, 6 h and 15 h. FIG. 12 shows that internalization of Tofa-NLC controls the secretion of pro-inflammatory cytokines reducing the extent of total accumulation.

FIG. 13. Oral administration of Tofa-NLC delays diabetes onset. We tested the effect of Tofa-NLC administration via oral gavage to two treatment groups: 1) 3 week-old NOD mice (corresponding to Pre-stage 1 in human patients) and 2) 10 week-old NOD mice (corresponding to advanced Stage 1 in human patients).

FIG. 14. Flow cytometry panels for CD44, FoxP3, FR4, CD73, CD49b, and LAG3 T-Cell markers used in the experimental methods to identify defined regulatory subsets.

FIG. 15. Graphs depicting accumulation of the anergic T-Cell subset (CD44^{hi}Foxp3-FR4^{hi}CD73^{hi}) in 10 week old female NOD mice that had been treated with Tofa-NLC at 3 week of age and in 12 week old female NOD mice that had been treated with Tofa-NLC at 10 week of age (same treatment groups used in FIG. 13), in pancreatic lymph nodes, mesenteric lymph nodes, inguinal lymph nodes (as control), spleen, and pancreas.

FIG. 16. Effective delivery and presentation of Bpep by NLC. Representative results of proliferation of Bpep-reactive TCR Tg T cells when stimulated by BMDC that were pre-exposed to soluble free peptide, Bpep-NLC, or nothing. Proliferation was assessed by CFSE staining of T cells before culture and assessment via flow cytometry of CFSE dilution. Colored histograms are interpolation of proliferation generated via ModFit software.

FIG. 17. Tofa-NLC release rate, as determined by HPLC.

FIG. 18. Incorporation kinetic of NLC by NOD mouse splenocytes.

Representative results of the incorporation of NLC-HLDI (1 μg/ml) by mouse splenocytes cocultured at 37° C. for the indicated time. Incorporation was measured as increased fluorescence in comparison to baseline (time 0). Each sub-population was identified via combination of pre-defined antibodies (CD3, CDTTb, CD11c, B220).

FIGS. 19A-19D. Distribution patterns of NLC. Representative live whole-animal images of fluorescent particles distribution after administration via different routes (using two different doses of HLDI-NLC). 19A) 4 hours post intravenous injection. 19B) 24 hours post oral gavage. 19C) 24 hours post intraperitoneal injection. 19D) 24 hours post subcutaneous injection.

FIG. 20. Kinetic of HLDI-NLC accumulation in specific lymphoid tissues.

Spleen, pancreatic lymph nodes, mesenteric lymph nodes, and inguinal lymph nodes were harvested 1, 2, 3 or 4 hours after oral administration of 15 mg NLC-HLDI and the fluorescence radiance quantified via IVIS.

FIG. 21. Lymph drainage along the length of the intestinal mucosa.

FIG. 22. Impact of lipid to surfactant ratio on particle size and polydispersity. Particle size (left) and PDI (right) initially decreased due to an increase in the surfactant ratio, but both resulted in slight quadratic trends. Analysis of particles was performed by dynamic light scattering. P values were determined from the All Pairs Tukey-Kramer Test.

FIG. 23. Screening of toxicity profile of Tofa-NLC formulations. Graph depicts the impact of different LNp formulations on the viability of mouse bone marrow-derived dendritic cells (DC). DC were cultured for 24 hours with titrations (numbers on top of each column, expressed as pg/ml of final lipid concentration) of different formulations of Tofa-NLC (indicated on right) and SLN (top row, used as reference). Numbers within each plot indicate the percentage of dying cells for the specific condition.

FIG. 24. Graphs depicting in changes in the antigen presenting cell's maturation marker CD80 in response to an in vivo challenge with the bacterial product LPS. This maturative stimulus was administered between the 4th and 5th administration of Tofa-NLC and its effect quantified the day after the last LNp administration (red dots) as well as in control animals that did not receibe LNp (blue triangles).

FIG. 25. An illustration depicting one proposed mechanism of therapeutic effect of the short term oral administration of Tofa-NLC of the present invention.

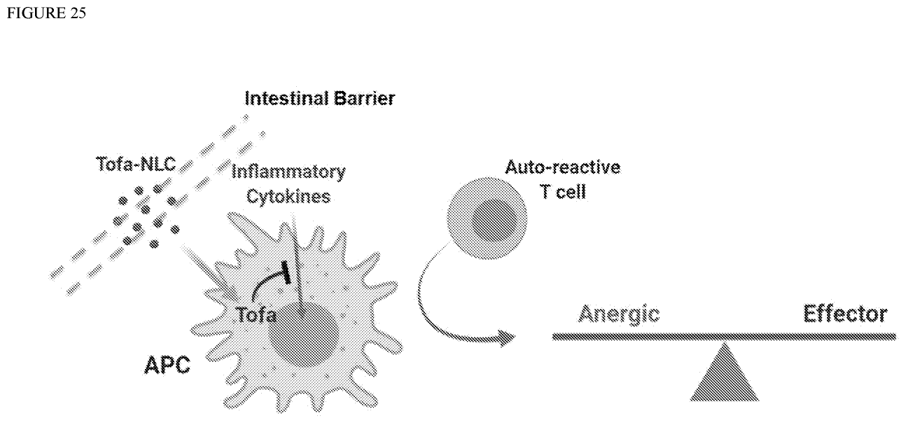

FIG. 26. A graph depicting Tofa release over time from different NLC formulations. The release of Tofa from the modified NLC formulations is affected by the surfactant composition and ratio.

### DETAILED DESCRIPTION OF THE INVENTION

The present invention provides compositions and methods for modulation of the interaction of T cell and DC to control the activation of auto-responses together with the engineering of LNp for controlled and localized co-delivery of a therapeutics and specific T1D autoantigen. The present invention is innovative in multiple respects: this is the first application of a targeted, biocompatible approach suitable for oral administration in the treatment of T1D. This is a transformative approach for the treatment of T1D patients. It is also the first time that the unique properties of LNp are employed for a combinatorial therapeutic strategy to target the lymphatics for the treatment of T1D, while minimizing the risk of toxic side effects. Building on the success of targeting the lymph nodes to improve the efficacy of vaccination,⁹² the inventive compositions and methods are positioned to promote effective regulation.

It will be understood by those of ordinary skill in the art that the lipid nanoparticles of the present invention can be prepared by using the Phase Inversion Temperature (PIT) method, although they can also be prepared using high energy methods such as ultrasonication, microfluidization, and high-pressure homogenization with similar or different components and/or reagents. First, the desired amount of drug, dye, or other cargo molecule is combined into a vial along with reagents to improve solubility, if necessary (e.g. ethanol, DMSO). The addition of lipids and then surfactants (by weight) follows. Then the mixture is co-melted and stirred (e.g. by vortexing). Lastly, purified water is added to the mixture, and heated to the phase inversion temperature until two phases (i.e. the aqueous and organic phases) are observed. Next, under continued stirring, cooling of the sample causes inversion of the water-in-oil emulsion to an oil-in-water emulsion; creating very small lipid droplets in the process and a transparent nanoemulsion is formed.

In some embodiments, the compositions comprising JAK inhibitors such as Tofa can be combined with checkpoint inhibitors, such as CTLA4-Ig, in the methods of the present invention.

The inventors have found that in some experimental results in a model of mouse heart transplantation that the transient combined use of CTLA4-Ig and Tofa has a profound and durable effect on the alloreactivity of T lymphocytes that would reject the graft. In particular, this effect is associated with a significantly higher accumulation of Treg in the graft in comparison to CTLA4-Ig alone. Moreover, this combination appears even more effective when employed in systems characterized by a heightened inflammatory state (ischemia in the present model).

Without being held to any particular theory, the inventors found the ability of Tofa to inhibit the production of inflammatory cytokines like IL-1 and TNF-α by maturing antigen presenting cells. Merged with the unique in vivo distribution property of LNp, it is thought that by combining the use of Tofa-LNp given orally, or other routes, with systemic CTLA4-Ig it will localize the actuation of enhanced costimulation blockade to lymphoid tissues that are involved in the activation and expansion of autoreactive T cells and will promote a lasting regulation.

The results presented herein clearly show that in the case of a mouse model of spontaneous T1D development, the short term oral administration of Tofa-NLC (without use of checkpoint modulators like CTLA4-Ig) was effective in delaying disease onset. It is also understood that this effect can be further enhanced with the concomitant localized delivery of T1D autoantigens (via LNp), as this will promote the actuation of enhanced costimulation blockade on antigen presenting cells presenting such antigens.

Thus, in accordance with an embodiment, the present invention provides a method of modulation of autoreactivity of T lymphocytes in a subject comprising administering to the subject a lipid nanoparticle composition comprising a JAK inhibitor.

As used herein, the term “JAK inhibitor” means a potent, selective inhibitor of the JAK family of tyrosine kinases, comprising four non-receptor tyrosine kinases, JAK1, JAK2, JAK3 and tyrosine kinase 2 (TYK2).

Examples of some JAK inhibitors include, but are not limited to Tofacitinib, Ruxolitinib, Decernotinib, Oclacitinib, Baricitinib, Peficitinib, Peficitinib, Fedratinib, Upadacitinib, Filgotinib, Cerdulatinib, Gandotinib, Lestaurtinib, Momelotinib, Pacritinib, and Abrocitinib.

In accordance with another embodiment, the present invention provides a method of modulation of autoreactivity of T lymphocytes in a subject comprising administering to the subject a lipid nanoparticle composition comprising Tofa

In accordance with another embodiment, the present invention provides a method of modulation of autoreactivity of T lymphocytes in a subject comprising administering to the subject a lipid nanoparticle composition comprising Tofa and a lipid nanoparticle composition comprising disease-related peptides.

As used herein, the term “disease related peptide” means defined fragments of proteins that activate autoreactive T and B lymphocytes as they are erroneously recognized as belonging to pathogens (examples: Insulin and Chromogranin A for Type 1 Diabetes; Myelin basic protein for Multiple Sclerosis; Citrullinated proteins for Reumathoid Arthritis; wheat gliadin and barley hordein for Celiac Disease).

In accordance with another embodiment, the present invention provides a method for prevention or treatment of Type 1 Diabetes in a subject comprising administering to the subject a lipid nanoparticle composition comprising Tofa and a lipid nanoparticle composition comprising diabetes targeted peptides (including for example peptides deriving from: insulin, glutamic acid decarboxylase 65 (GADA), islet-antigen-2 (IA-2A), zinc transporter 8 (ZnT8A), and hydrid peptides—newly recognized peptides formed by the fusion of peptides of the aforementioned proteins).

By “ameliorate” is meant decrease, suppress, attenuate, diminish, arrest, or stabilize the development or progression of a disease.

By “alteration” is meant a change (increase or decrease) in the expression levels of a gene or polypeptide as detected by standard art known methods such as those described above. As used herein, an alteration includes a 10% change in expression levels, preferably a 25% change, more preferably a 40% change, and most preferably a 50% or greater change in expression levels.”

“Biological sample” as used herein refers to a sample obtained from a biological subject, including sample of biological tissue or fluid origin, obtained, reached, or collected in vivo or in situ, that contains, or is suspected of containing, nucleic acids or polypeptides. Such samples can be, but are not limited to, organs, tissues, fractions and cells isolated from mammals including, humans such as a patient, mice, and rats. Biological samples also may include sections of the biological sample including tissues, for example, frozen sections taken for histologic purposes.

By “disease” is meant an immune disorder and more particularly, an autoimmune disorder. As used herein, the term “autoimmune disease” means a disease resulting from an immune response against a self-tissue or tissue component, including both self-antibody responses and cell-mediated responses. The term autoimmune disease, as used herein, encompasses organ-specific autoimmune diseases, in which an autoimmune response is directed against a single tissue, such as type I diabetes mellitus (T1D), Crohn's disease, ulcerative colitis, myasthenia gravis, vitiligo, Graves' disease, Hashimoto's disease, Addison's disease and autoimmune gastritis, autoimmune hepatitis, primary biliary cirrhosis, and autoimmune thrombocytopenia. The term autoimmune disease also encompasses non-organ specific autoimmune diseases, in which an autoimmune response is directed against a component present in several or many organs throughout the body. Such autoimmune diseases include, for example, rheumatoid diseases, systemic lupus erythematosus, progressive systemic sclerosis and variants, polymyositis and dermatomyositis, inflammatory bowel disease, celiac disease, inflammatory myositis, Sjogren's syndrome, multiple sclerosis, psoriasis and scleroderma.

In addition it is contemplated that the compositions and methods of the present invention would also be useful in the immune-related Graft-versus-Host-disease as a result of bone marrow transplantation.

By “an effective amount” is meant the amount required to identify, diagnose, image, or ameliorate the symptoms of a disease relative in an untreated or treated patient. The effective amount of active compound(s) used to practice the present invention for therapeutic treatment of a neurodegenerative disease varies depending upon the manner of administration, the age, body weight, and general health of the subject. Ultimately, the attending physician or veterinarian will decide the appropriate amount and dosage regimen. Such amount is referred to as an “effective” amount.

The data obtained from the cell culture assays and animal studies can be used in formulating a range of dosage for use in humans. The dosage of such antibodies or antigen-binding fragments thereof lies generally within a range of circulating concentrations of the antibodies or fragments that include the ED₅₀ with little or no toxicity. The dosage may vary within this range depending upon the dosage form employed and the route of administration utilized. A therapeutically effective dose can be estimated initially from cell culture assays. A dose can be formulated in animal models to achieve a circulating plasma concentration range that includes the IC₅₀ (i.e., the concentration of the antibody which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration is desired, cell culture or animal modeling can be used to determine a dose required to achieve a therapeutically effective concentration within the local site.

The biologically active agents can include immunotherapeutic agents. The term “immunotherapeutic agent” can include any molecule, peptide, antibody or other agent which can stimulate a host immune system to generate an immune response to a tumor or cancer in the subject. Various immunotherapeutic agents are useful in the compositions are known in the art and include, e.g., PD-1 and/or PD-1L inhibitors, CD200 inhibitors, CTLA4 inhibitors, and the like. Exemplary PD-1/PD-L1 inhibitors (e.g., anti-PD-1 and/or anti-PD-L1 antibodies) are known in the art and described in, e.g., International Patent Application Publication Nos. WO 2010036959 and WO 2013/079174, as well as U.S. Pat. Nos. 8,552,154 and 7,521,051, the disclosures of each of which as they relate to the antibody descriptions are incorporated herein by reference in their entirety. Exemplary CD200 inhibitors are also known in the art and described in, e.g., International Patent Application Publication No. WO 2007084321. Suitable anti-CTLA4 antagonist agents are described in International Patent Application Publication Nos. WO 2001/014424 and WO 2004/035607; U.S. Patent Application Publication No. 2005/0201994; and European Patent No. EP 1212422. Additional CTLA-4 antibodies are described in U.S. Pat. Nos. 5,811,097, 5,855,887, 6, 051, 227, and 6,984,720. It is understood that the immunomodulatory agents can also be used in conjunction with a compound described herein for the treatment of an infection, such a viral, bacterial, or fungal infection, or any other condition in which an enhanced immune response to an antigen of interest would be therapeutically beneficial.

In some embodiments, the NLp and NLC which comprise disease-specific antigen peptides or small molecules can be labeled.

As used herein, the term “labeled” means a compound or protein or peptide or other biologically active molecule which has a detectable moiety linked to it either covalently or via a linking molecule.

By “detectable label(s) or moieties” is meant a composition that when linked to a molecule of interest renders the latter detectable, via spectroscopic, photochemical, biochemical, immunochemical, chemical means or other imaging means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. Specific radioactive labels include most common commercially available isotopes including, for example, ³H, ¹¹C, ¹³C, ¹⁵N, ¹⁸F, ¹⁹F, ¹²³I, ¹²⁴I ¹²⁵I, ¹³¹I, ⁸⁶Y, ⁸⁹Zr, ¹¹¹In, ^{94m}Tc, ^{99m}Tc, ⁶⁴Cu and ⁶⁸Ga. Suitable dyes include any commercially available dyes such as, for example, 5(6)-carboxyfluorescein, IRDye 680RD maleimide or IRDye 800CW, ruthenium polypyridyl dyes, and the like. Also included in the labeled substrates of the present invention are substrates labeled with PET, SPECT or MRI detectable imaging agents or moieties.

In accordance with an embodiment, the detectable moiety is a fluorescent dye. The dyes may be emitters in the visible or near-infrared (NIR) spectrum. Known dyes useful in the present invention include carbocyanine, indocarbocyanine, the far-red dye 1, 1′,3,3,3′, 3′-Hexamethylindodicarbocyanine Iodide, oxacarbocyanine, thuicarbocyanine and merocyanine, polymethine, coumarine, rhodamine, xanthene, fluorescein, boron˜dipyrromethane (BODIPY), Cy5, Cy5.5, Cy7, VivoTag-680, VivoTag-5680, VivoTag-5750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

Organic dyes which are active in the NIR region are known in biomedical applications. However, there are only a few NIR dyes that are readily available due to the limitations of conventional dyes, such as poor hydrophilicity and photostability, low quantum yield, insufficient stability and low detection sensitivity in biological system, etc. Significant progress has been made on the recent development of NIR dyes (including cyanine dyes, squaraine, phthalocyanines, porphyrin derivatives and BODIPY (borondipyrromethane) analogues) with much improved chemical and photostability, high fluorescence intensity and long fluorescent life. Examples of NIR dyes include cyanine dyes (also called as polymethine cyanine dyes) are small organic molecules with two aromatic nitrogen-containing heterocycles linked by a polymethine bridge and include Cy5, Cy5.5, Cy7 and their derivatives. Squaraines (often called Squarylium dyes) consist of an oxocyclobutenolate core with aromatic or heterocyclic components at both ends of the molecules, an example is KSQ-4-H. Phthalocyanines, are two-dimensional 18πr-electron aromatic porphyrin derivatives, consisting of four bridged pyrrole subunits linked together through nitrogen atoms. BODIPY (borondipyrromethane) dyes have a general structure of 4,4′-difluoro-4-bora-3a, 4a-diaza-s-indacene) and sharp fluorescence with high quantum yield and excellent thermal and photochemical stability.

Other imaging agents which can be used in the labeled substrates of the present invention include PET and SPECT imaging agents. The most widely used agents include branched chelating agents such as di-ethylene tri-amine penta-acetic acid (DTPA), 1,4,7,10-tetra-azacyclododecane-1,4,7,10-tetraacetic acid (DOTA) and their analogs. Chelating agents, such as di-amine dithiols, activated mercaptoacetyl-glycyl-glycyl-gylcine (MAG3), and hydrazidonicotinamide (HYNIC), are able to chelate metals like ^{99m}Tc and ¹⁸⁶Re. Instead of using chelating agents, a prosthetic group such as N-succinimidyl-4-¹⁸F-fluorobenzoate (¹⁸F-SFB) is necessary for labeling peptides with ¹⁸F.

In accordance with an embodiment, the detectable moiety may be attached to the substrate by a linker molecule. For instance linking groups having alkyl, aryl, combination of alkyl and aryl, or alkyl and aryl groups having heteroatoms may be present. For example, the linker can be a C₁-C₂₀ alkyl, C₂-C₂₀ alkenyl, C₂-C₂₀ alkynyl, C₁-C₂₀ hydroxyalkyl, C₁-C₂₀ alkoxy, C₁-C₂₀ alkoxy C₁-C₂₀ alkyl, C₁-C₂₀ alkylamino, di-C₁-C₂₀ alkylamino, C₁-C₂₀ dialkylamino C₁-C₂₀ alkyl, C₁-C₂₀ thioalkyl, C₂-C₂₀ thioalkenyl, C₂-C₂₀ thioalkynyl, C₆-C₂₂ aryloxy, C₆-C₂₂ arylamino C₂-C₂₀ acyloxy, C₂-C₂₀ thioacyl, C₁-C₂₀ amido, and C₁-C₂₀ sulphonamido.

In some embodiments, the one or more small molecule inhibitors and/or biologically active agents in the NLp are labeled with one or more detectable moieties.

The compositions can take the form of solutions, suspensions, emulsions, powders, sustained-release formulations, depots and the like. Examples of suitable carriers are described in “Remington's Pharmaceutical Sciences,” Martin. Such compositions will contain an effective amount of the biopolymer of interest, preferably in purified form, together with a suitable amount of carrier so as to provide the form for proper administration to the patient. As known in the art, the formulation will be constructed to suit the mode of administration.

Generally, the ingredients are supplied either separately or mixed together in unit dosage form, for example, as a dry lyophilized powder or water-free concentrate in a sealed container, such as an ampule or sachet indicating the quantity of active agent. Where the composition is to be administered by infusion, it can be dispensed with an infusion bottle containing sterile pharmaceutical grade water or saline. Where the composition is administered by injection, an ampule of sterile water for injection or saline can be provided, for example, in a kit, so that the ingredients may be mixed prior to administration.

The carriers or diluents used herein may be solid carriers or diluents for solid formulations, liquid carriers or diluents for liquid formulations, or mixtures thereof.

Solid carriers or diluents include, but are not limited to, extracellular matrix, any scaffolds, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

In some embodiments, gelucires are used. Gelucires are polyethylene glycol (PEG) glycerides composed of mono-, di- and triglycerides and mono- and diesters of PEG. They are inert semi-solid waxy amphiphilic excipients with surface-active properties that spontaneously form a fine dispersion or emulsion upon contact with water.

For liquid formulations, pharmaceutically acceptable carriers may be, for example, aqueous or non-aqueous solutions, or suspensions. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Intravenous vehicles include, for example, fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions.

The choice of carrier will be determined, in part, by the particular compositions, as well as by the particular method used to administer the composition. Accordingly, there are a variety of suitable formulations of the pharmaceutical compositions of the invention. The following formulations for parenteral, subcutaneous, intravenous, intramuscular, intraarterial, intrathecal and interperitoneal administration are exemplary, and are in no way limiting. More than one route can be used to administer the compositions of the present invention, and in certain instances, a particular route can provide a more immediate and more effective response than another route.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., autoimmune disease being treated or prevented. Also, for purposes herein, “prevention” can encompass delaying the onset of the disease, or a symptom or condition thereof.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

### EXAMPLES

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

Composition of Tofa-NLC formulation:

Solid Lipid: Tetracosane (C24).

Liquid lipid: Tocopherol (pubchem.ncbi.nlm.nih.gov/compound/alpha-Tocopherol). Surfactant: Gelucire (gattefosse.com/gelucire-4414 and similar).

Processing of NLC.

NLCs were prepared using a method that keeps the composition constant while the temperature is changed. This method is called phase inversion temperature (PIT) method^{65, 66, 70, 87}. This versatile method allows the synthesis of small sizes and low polydispersity lipid nanoparticles with controlled latent heat of melting and melting point.

Particle Size and Polydispersity.

The particle size and the PDI of the NLCs were measured using a Malvern Zetasizer Nano S dynamic light scattering system. In this technique, the frequency of the shifted light is used to measure the particle size. The particle size of the NLCs was measured after adding 25 μL of particles into 3 mL of water. The water was filtered with a 0.2 μm filter directly into a cuvette prior the addition of the NLCs. The particle diameter and PDI of particle size distribution are reported. Each formulation of the DOE was measured three times.

Melting Point and Latent Heat of Melting.

The thermal behavior of NLCs was studied using a Mettler-Toledo differential scanning calorimeter (DSC) equipped with an auto-sampler and liquid nitrogen as the cooling source. The as-prepared NLCs were pipetted into a 40 μL aluminum pan with a mass of approximately 25 mg. Then, the aluminum pan was hermetically sealed to minimize moisture loss during the DSC scan. The NLCs were measured from 5 to 80° C. The latent heat of melting was calculated using the integral of the area under the curve (amount of energy) divided by the amount of material and the valley in the DSC plot represents the melting point. The melting point and the latent heat of melting are reported.

Tofa Encapsulation Efficiency and Release.

Determination of the Tofa-NLC drug loading, or encapsulation efficiency (EE %) was calculated following separation of the organic and aqueous phases using a spin-X UF concentrator (MWCO=5K) followed by centrifugation. Following this separation, the final volumes for both phases were analyzed using HPLC to quantify the amount of Tofa in each phase. Freshly prepared Tofa-NLCs were analyzed in parallel to quantify the total amount of Tofa in each starting preparation. EE was calculated by dividing the mass of Tofa in the organic phase by the total mass of Tofa and multiplying by 100. Three independent formulations were prepared and tested by this method.

The release kinetics of Tofa-NLC were determined following dialysis of a 200 μL sample of each freshly prepared Tofa-NLC sample into a 5 ml volume of phosphate buffered saline (37° C., shaking) for a given time period. Three technical replicates of each sample were prepared and analyzed over a 72-hour time course. At specific time points, an aliquot of the buffer was collected for subsequent HPLC analysis.

HPLC Method for Tofa.

The mobile phase for analysis was 1-Octanesulfonic acid with 0.1% Phosphoric Acid (H₃PO₄) and Acetonitrile (75:25, v/v). The analysis was performed isocratically with a flow rate of 0.25 mL/min. The detection wavelength was set to 290 nm.

The 1-Octanesulfonic acid was used as diluent for sample and standards preparations. Each volume of accurately weighed Tofa standard sample was diluted to volume with diluent to obtain a standard curve. The concentrations of the standard curve were 40 μg/mL, 20 μg/mL, 6 μg/mL, 0.6 μg/mL, and 0.072 μg/mL. The R² value for the best fit line for the calibration curve had a value of 1.000. The system proved to be suitable for the analysis with a % RSD value of replicate injections of the 40 μg/mL standard solution of 0.41.

Each accurately measured aliquot of the separated NLC solutions, containing Tofa, was diluted to volume with diluent and run on HPLC using the exact parameters as above. All samples were injected using a 20 μL sample loop. Tofa eluted at approximately 2.04 minutes for all samples and standards injected. The complete run time for all chromatographic runs was 6.0 minutes.

### Example 1

Targeted Jak inhibition synergizes with CoB to prolong transplant survival.

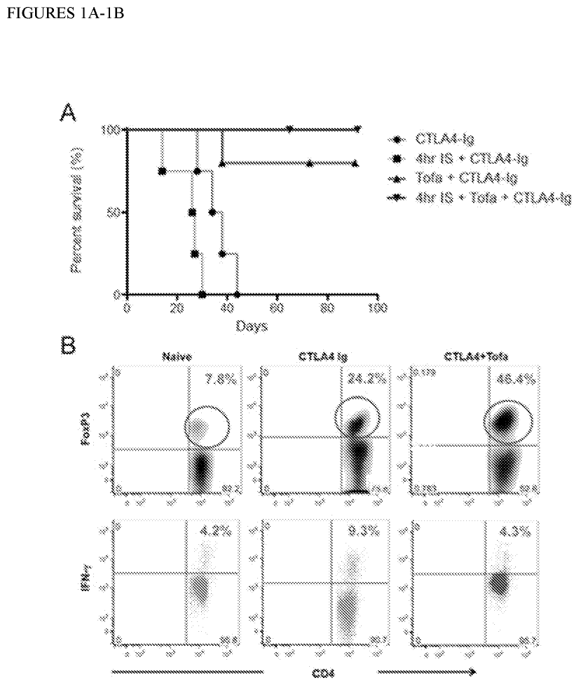

The feasibility of combining CTLA4-Ig with a short-course Tofa administration in a full MHC mismatch (C₅₇BL/6 to BALB/c) mouse heart transplantation model was tested (FIG. 1A). Encouragingly, heart transplant survival in mice treated with CTLA-4Ig was significantly improved by a short course (10 days) of Tofa (MST untreated: 9d, CTLA4-Ig only: 36d, Tofa+CTLA4-Ig: 120d). This improvement was evident even in mice receiving hearts kept ischemic for 4 h before transplantation—a more clinically relevant scenario—where CTLA4-Ig efficacy is undermined by early accumulation of inflammatory mediators⁶⁷ (MST untreated: 9d, CTLA4-Ig only: 26.5d, Tofa+CTLA4-Ig: 150d). Analysis of the graft infiltrate by flow cytometry revealed that the protective effect of the combination of Tofa+CTLA4-Ig was associated the accumulation of a much higher frequency of Treg cells (FIG. 1B). Moreover, ex-vivo restimulation of splenic T lymphocytes showed that Tofa+CTLA4-Ig inhibited the generation of IFN-γ producing (Th1) T cell. These results indicate that pharmacological inhibition of inflammatory cytokines (via Tofa) is particularly effective in synergizing with CTLA4-Ig to effectively modulate the activation of reactive T cells and promoting transplant survival—a strategy that we expect can be optimized for establishing proper immune modulation of autoreactivity.

### Example 2

Targeted Jak inhibition limits DC maturation.

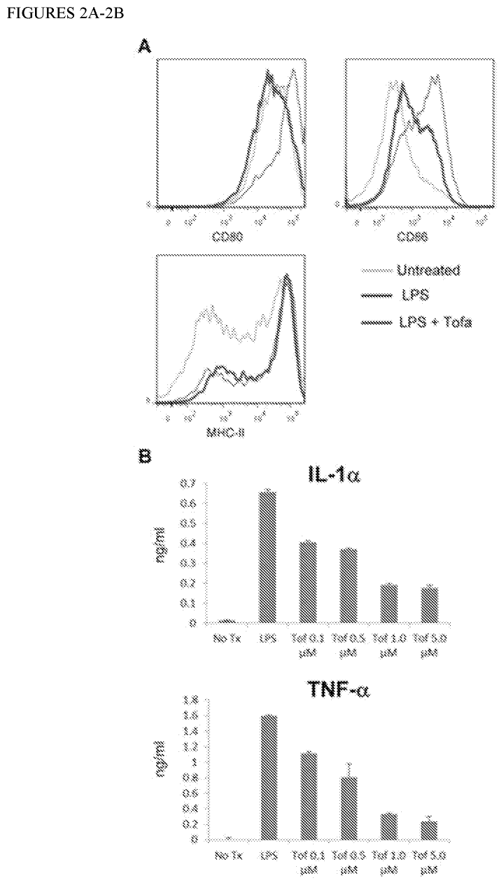

To better understand the impressive protective effect achieved with the combination Tofa+CTLA4-Ig, we tested the ability of Tofa to inhibit the maturation of bone marrow derived dendritic cells (DC) in response to LPS (FIG. 2). Exposure to Tofa caused a significant inhibition of the up-regulation of CD80 and CD86 costimulatory molecules, with a concomitant reduction in release of the prototypical inflammatory cytokines IL-6 (not shown), IL-1, and TNF-α. The latter two have been implicated in T1D development and inhibition of their accumulation would be a valuable effect. Interestingly, Tofa did not prevent the up-regulation of MHC-II molecules, rendering DC with high Signal 1 but very low Signal 2 (costimulatory molecules) and low Signal 3 (cytokines)—a phenotype that could maximize the impact of CTLA4-Ig on reactive T cells.

### Example 3

Tofa does not interfere with Treg suppression of T cells.

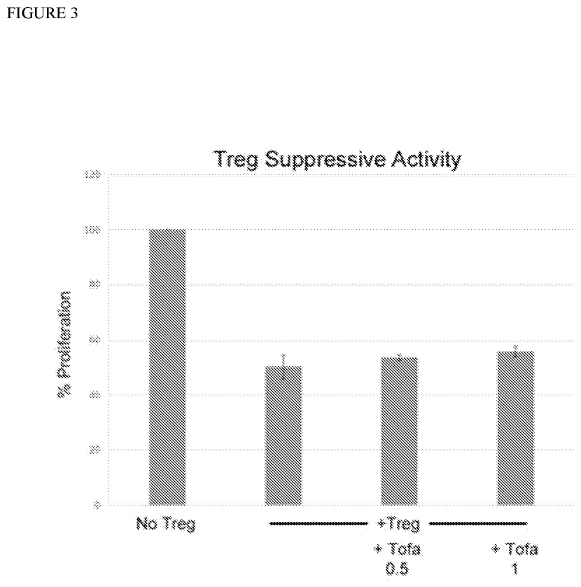

The observed accumulation of Treg in transplants treated with Tofa+CTLA4-Ig warranted confirmation that Tofa (although transient) would not interfere with Treg function. The results of a T cell CFSE-Proliferation-Assay conducted to assess the suppressive activity of Treg (added at either 2:1 or 4:1 T cell to Treg ratio) without and with Tofa (at 0.5 or 1 μM) confirmed the preservation of regulatory activity (FIG. 3). These results show the potential of the combined use of Tofa with CTLA4-Ig for induction of long-term regulation.

### Example 4

Optimization of LNp synthesis for permeation of biological barriers and therapeutic delivery of Tofa to immune cells.

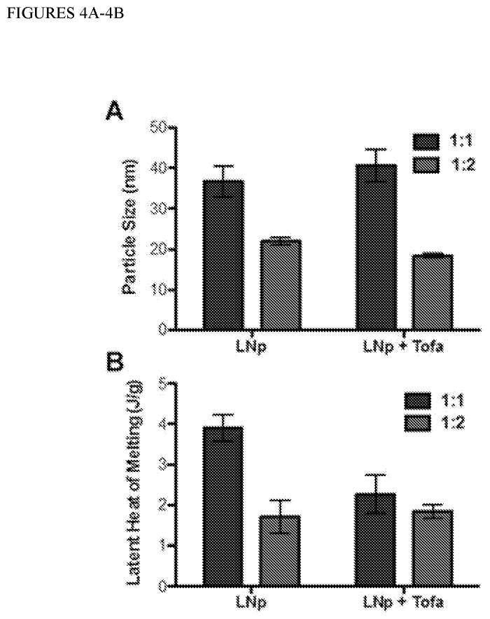

Solid lipid nanoparticles (SLNs—one of the LNp types used) are comprised of solid spheres of lipid surrounded by nonionic surfactants, and can be loaded with a variety of molecules (e.g. Tofa).⁶⁵ Unlike the many complex methods used for SLN production, the inventors optimized a facile synthesis process for biocompatible drug delivery vehicles, based on the phase inversion temperature (PIT) method.⁶⁸⁻⁷⁰ The PIT method increases clinical translatability by allowing precise control over particle characteristics such as chemical composition, size, polydispersity, and thermal behavior. These properties have been shown to have a dramatic effect on cellular interactions, influencing uptake efficiency, internalization pathways, intracellular localization, and cytoxicity.⁷¹⁻⁷⁵ Similarly, particle crystallinity has been shown to be an important factor affecting the drug entrapment and delivery efficiency of incorporated therapeutic molecules.⁷⁶ Using this synthesis process, we demonstrated the influence of specific parameters, such as the lipid to surfactant ratio, in altering these properties. By constraining the synthesis variables, we optimized the production of sub-30 nm sized, non-toxic SLNs that are particularly suited to penetrate biological barriers like the intestinal wall (see Examples 14 and 15 below).^{66, 77} We then examined the impact of several process variables on the formulation of LNp containing Tofa (FIG. 4). The use of an increased lipid to surfactant ratio (1:2) for example, resulted in smaller particles with a decreased latent heat of melting, properties favorable for penetration of biological barriers (less than 30 nm) and maximal drug loading capacity (disordered crystal structure). The incorporation and stability of Tofa within the particles was measured by high performance liquid chromatography (HPLC) where 100% recovery of the calculated Tofa concentration was demonstrated with no indication of drug degeneration (not shown).

### Example 5

Tofa-LNp modulate DC's antigen presenting function.

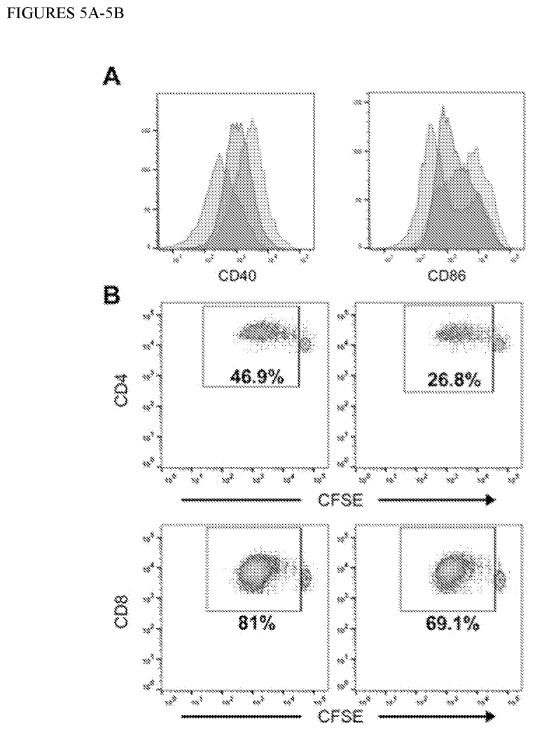

We tested whether the exposure of DC to Tofa-LNp (and empty-LNp as control), for either 18 h or 42 h, had an effect on DC's functions comparable to that of soluble Tofa. First, we determined whether this exposure influenced the upregulation of the maturation markers CD40, CD80, CD86, and MHC-II (assessed by flow cytometry) induced by the maturative stimuli LPS. The data clearly show that 18 h of exposure to Tofa-LNp was sufficient to significantly prevent the up-regulation of these maturative markers. However, it was noted that an exposure of 42 h exerted an even more robust inhibition (FIG. 5A). With these encouraging results, we assessed the T cell stimulatory capacity of Empty-Vs Tofa-LNp conditioned DC (either before or after exposure to LPS) in conventional MLR (using CFSE-labeled allogeneic T cells). The results indicated that Tofa-LNp conditioned DC were less stimulatory than Empty-LNp conditioned DC (FIG. 5B). These results confirmed that Tofa-LNp are effective at delivering their cargo to DC.

### Example 6

LNp biodistribution and uptake.

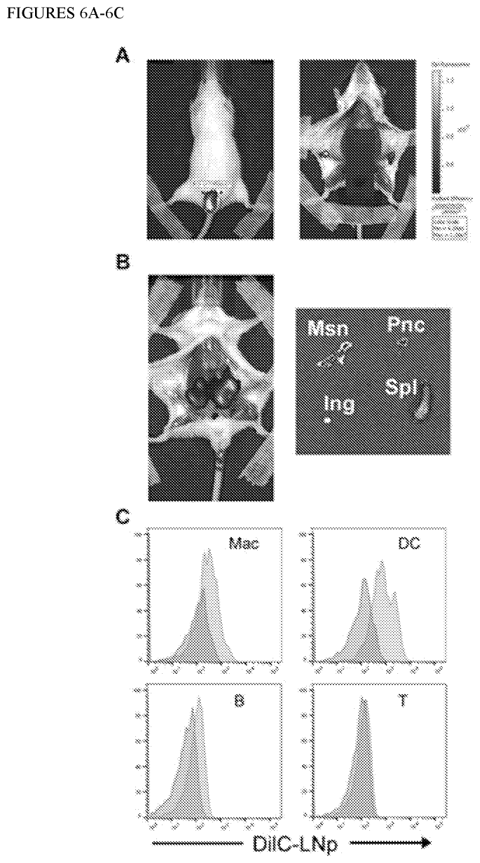

To study the impact that differential routes of Tofa-LNp administration have on in vivo absorption, distribution, and pharmacokinetics we manufactured LNp containing the far-red dye 1, 1′,3,3,3′, 3′-Hexamethylindodicarbocyanine Iodide (HLDI-LNp). We employed a Perkin Elmer IVIS Spectrum system for non-invasive optical imaging to determine the tissue distribution of injected HLDI-LNp at multiple time points (hours to days) in the same cohort of animals. We focused on three routes of LNp administration: 1) subcutaneous injection, 2) intraperitoneal injection, and 3) oral gavage. Our results confirmed that LNp have the unique property to accumulate in lymphoid tissues, with a tropism dictated by the route of administration. For example, following subcutaneous injection at the base of the tail, LNp accumulated exclusively in the inguinal lymph nodes (FIG. 6A). Differently, following intraperitoneal injection, LNp accumulated exclusively in the mesenteric lymph nodes (data not shown). In the case of oral gavage, the strongest accumulation occurred in the mesenteric lymph nodes, the pancreatic lymph nodes, and the spleen FIG. 6B). We started characterizing the LNp uptake by immune populations in the tissues identified by the IVIS system (e.g. inguinal lymph nodes for s.c. injection). All cells with antigen presenting capacity (macrophages, DC, and B cells) showed significant LNp uptake, although to different extents (FIG. 6C).

Overall, these data indicate that implementation of therapeutic-loaded LNp is an effective approach to achieve the spatial and temporal targeting of immune cell populations necessary to maximize Tofa/CTLA4-Ig and autoantigen synergism and minimize side effects.

### Example 7

Demonstrate targeted delivery and presentation of insulin autoantigens via an optimized LNp formulation.

Insulin B peptide-loaded LNp (Bpep-LNp) will improve the stability and bioavailability of this T1D autoantigen providing a controlled and targeted delivery, following oral administration, to antigen presenting cells in lymphoid tissues draining the gut.

Owing to the complexity of oral insulin delivery, numerous approaches have been taken to increase its bioavailability including microemulsion, liposomal, and nanoparticle formulations.⁸² Despite the limited solubility of intact insulin in lipid matrices, several groups have successfully formulated insulin-LNp for use as a T1D treatment.^{50, 83-86} Furthermore, liposomal delivery of insulin peptides has been demonstrated to have tolerogenic effects in a NOD mouse model.⁴² This is particularly relevant, as the encapsulation of insulin peptides improves their stability while also preventing the use of endocrinologically active insulin that would have serious side effects. Based on our extensive experience in LNp formulations, and the results obtained with Tofa-LNp, we believe that encapsulation of the insulin B peptide (sequence B9-23) will be readily obtainable resulting in Bpep-LNp that will deliver this autoantigen specifically to antigen presenting cells in a way that maximizes its use for antigen specific immunotherapy.

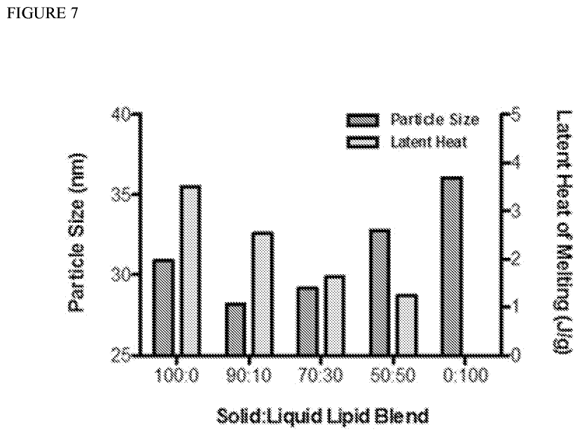

LNp is formulated using the PIT method^{66, 87}. We have previously optimized the lipid type and the lipid to surfactant ratio for synthesis of sub-30 nm Tofa-LNp. A similar strategy is employed for insulin-LNp formulation. Similar to SLN (used for Tofa), nanostructured lipid carriers (NLC) can be exploited to further improve the stability of the proposed technology platform. NLCs have long-term stability and higher therapeutic loading capacity. Our preliminary results have shown the ability of the PIT method to synthesize NLCs with varied particle size, polydispersity and thermal behavior (FIG. 7). The synthesis of NLCs resulted in particles with a low level of crystallinity, which represents increased peptide loading potential and stability.

### Example 8

Functional characterization of novel formulation of Tofa-NLC.

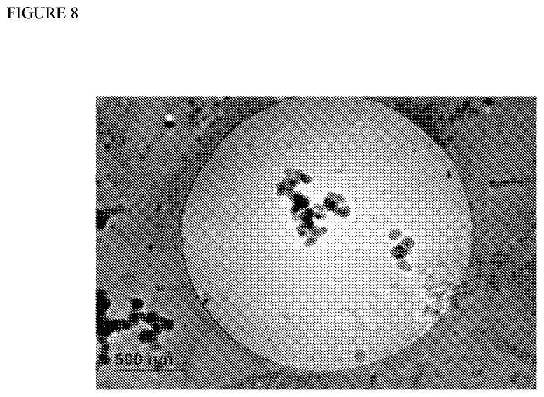

The present invention provides a formulation, Tofa-NLC #6, with a very promising low toxicity profile and that is going to be subjected to additional cycles of optimization and then implemented in both our in vitro and in vivo studies. To characterize the stability of this low-toxicity LNp formulation, we prepared three separate batches of LNp for particle size analysis. Dynamic light scattering (DLS) was utilized to quantitate particle size and polydispersity. Minimal change in average particle size was observed over the course of two weeks (Table 1). In addition, we have acquired images of the NLC using transmission electron microscopy (TEM; FIG. 8). Particles were observed to be consistent with the average size measurements previously determined by DLS.

TABLE 1
Particle size over time.
Particle Size (nm)

| Replicate | Time 0 | Week 1 | Week 2 |
|---|---|---|---|
| 1 | 54.1 | 70.9 | 79.6 |
| 2 | 83.6 | 69.8 | 78.9 |
| 3 | 85.4 | 76.8 | 91.5 |
| Average | 71 ± 15 | 72.5 ± 4 | 83.3 ± 7 |

NLC particles were characterized over time using dynamic light scattering to assess particle size. Three independent replicates were measured.

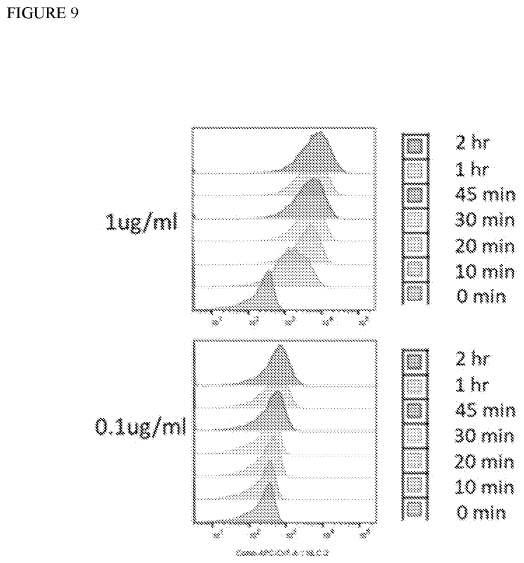

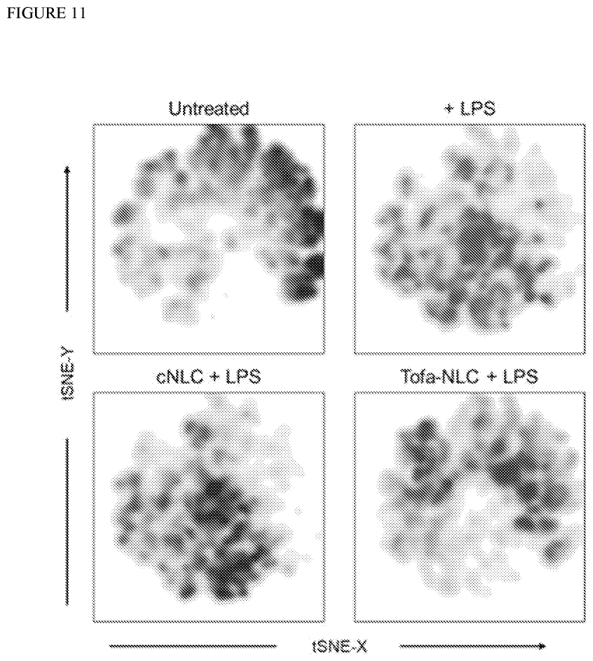

Using the same lipid/surfactant formulation of the newly characterized NLC (without Tofa) loaded with the fluorescent dye HLDI, we studied the incorporation kinetic of these particles by mouse bone marrow-derived dendritic cells (BMDC). As shown in FIG. 9, HLDI-NLC are rapidly incorporated by BMDC with a function that is proportional to their concentration and to the length of exposure. Having confirmed incorporation, we proceeded in assessing the ability of Tofa-NLC to deliver Tofa to BMDC in an amount sufficient to affect their maturation in response to the danger signal LPS (bacterial lipopolysaccharide). As measured by inhibition of maturation markers expression via flow cytometry, a short (4-6 h) pre-exposure of BMDC to Tofa-NLC was sufficient to significantly prevent the extent of up-regulation of the markers assessed (FIG. 11 (10), showing a cumulative representation of the maturation status of BMDC, via t-SNE plot, in the various conditions tested). We are currently in the process of assessing alterations in the pro- and anti-inflammatory cytokines content of the supernatant of these BMDC cultures via Luminex assay.

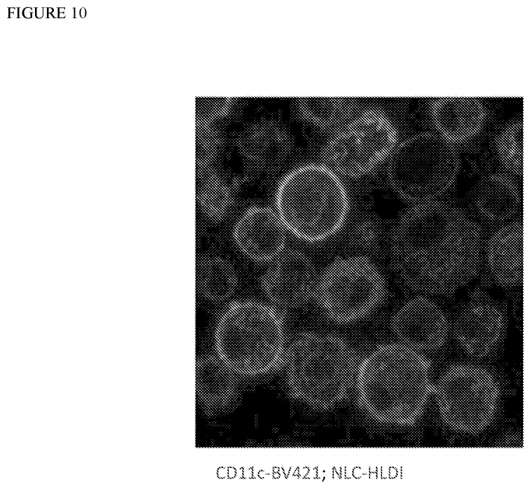

The observed kinetic of NLC incorporation by BMDC (FIG. 9) could result from a true internalization of the particles by the cells or by adherence of the particles to the extracellular membrane. We performed confocal microscopy analysis of HLDI-NLC exposed BMDC (1 μg/ml particles concentration, 10 minutes incubation time) to identify the cellular localization of the nanoparticles. The results, FIG. 10, clearly show that the NLC (red) are internalized by mouse BMDC (cyan) and accumulate intracellularly.

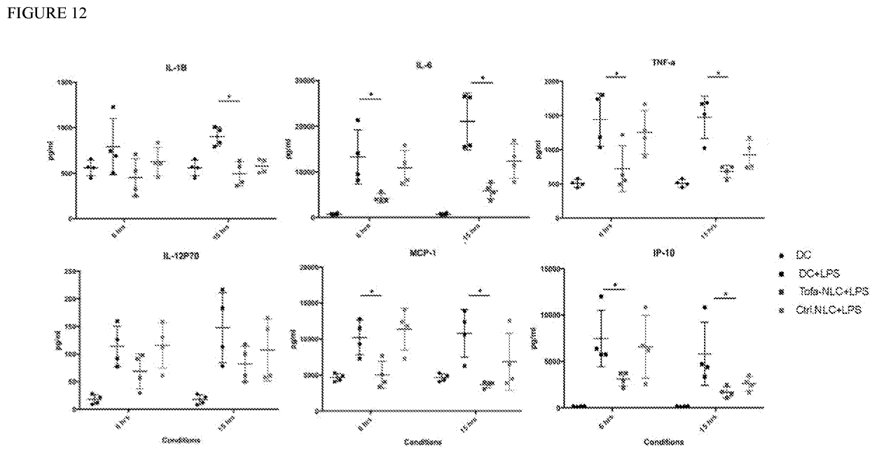

In addition to assessing the maturation inhibition of mouse BMDC by the internalization of Tofa-NLC, we determined via Luminex technology changes in the accumulation of pro-inflammatory cytokines by maturing cells in the same treatment groups as FIG. 11. Two time points post addition of the stimulus (LPS) were analyzed, 6 h and 15 h. FIG. 12 shows that internalization of Tofa-NLC controls the secretion of pro-inflammatory cytokines reducing the extent of total accumulation.

### Example 9

Oral administration of Tofa-NLC delays diabetes onset.

Having confirmed the capacity of the new Tofa-NLC formulation to deliver their cargo, we assessed whether administration of these LNp in vivo would have any toxic effect. 10-week old NOD mice were treated with 5 administrations via oral gavage, one every other day, of 15 mg/mouse Tofa-NLC (carrying a total of 200 μg Tofa/administration) and followed overtime. No sign of weight loss or other distress was observed showing the in-vivo safety of Tofa-NLC.

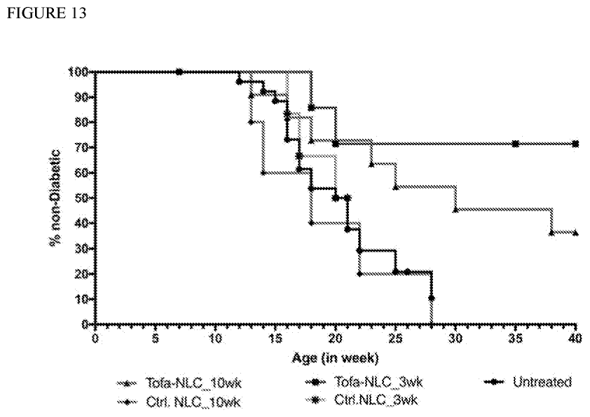

All these characteristics indicate that LNp would be a most valuable delivery system for the tissue selective actuation of a combination immunotherapy. We then began to define the effects of a transient administration of Tofa-NLC on diabetes development in NOD mice. As the conventional regimen of CTLA4-Ig administration in mouse models of transplantation is 4 every other day injections, we speculated that 5 every other day administrations of Tofa-NLC would provide a localized and sustained release of Tofa that would parallel the presence of CTLA4-Ig (based on the Tofa release profile observed). We then tested the effect of Tofa-NLC administration via oral gavage to two treatment groups: 1) 3 week-old NOD mice (corresponding to Pre-stage 1 in human patients) and 2) 10 week-old NOD mice (corresponding to advanced Stage 1 in human patients). Surprisingly, despite the absence of CTLA4-Ig or peptide administration, both treatments had a profound beneficial effect (FIG. 13). In particular, oral administration to 3-week-old NOD mice prevented onset of disease in 70% of the animals for more than 40 weeks. Transient treatment of 10 week-old female NOD mice also provided a statistically significant improvement on disease development, though not to the extent obtained with 3 week-old mice. Administration of empty NLC as control did not provide any appreciable effect. More importantly, the currently ongoing group treated with free Tofa (providing an amount identical to that used in the formulation of Tofa-NLC) already indicates an inferior (if not absent) therapeutic effect. This result suggests that the localized delivery achieved via LNp enables the actuation of immunoregulatory effects that are not otherwise achievable with more conventional approaches.

### Example 10

Short term oral administration of Tofa-NLC promotes the accumulation of anergic T cells.

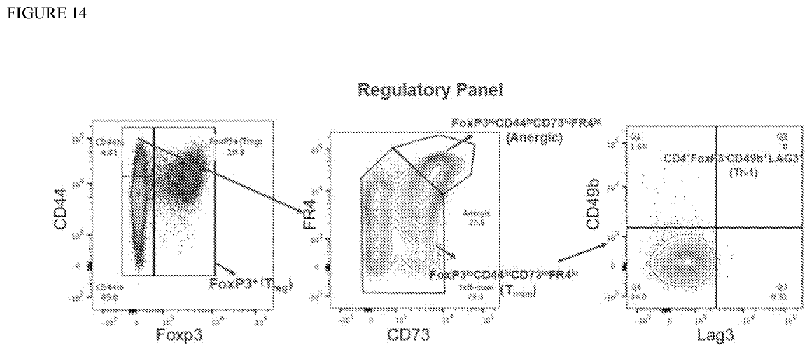

Multiple types of regulatory T cells have been involved in the prevention of autoimmunity. Of these, three subsets are consistently involved in the regulation of diabetes development: Treg, TR1, and anergic T cells.^{5, 76, 80, 81} We have then developed a flow cytometry panel to identify the tissue-specific abundance of these three subsets (FIG. 14).

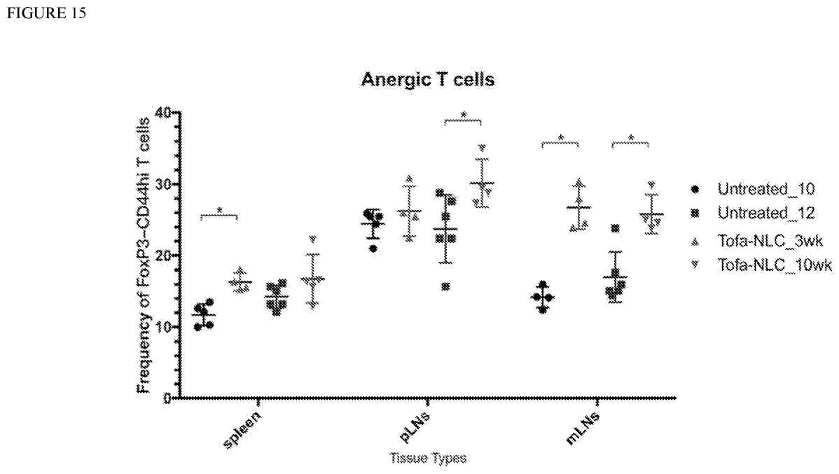

As part of our broader project to develop LNp-based therapies of immunoregulation, we analyzed the accumulation of these three subsets in 10 week old female NOD mice that had been treated with Tofa-NLC at 3 week of age and in 12 week old female NOD mice that had been treated with Tofa-NLC at 10 week of age (same treatment groups used in FIG. 13). We analyzed the composition of the T cell pool in pancreatic lymph nodes, mesenteric lymph nodes, inguinal lymph nodes (as control), spleen, and pancreas. Of the three populations quantified, the results show that Tofa-NLC treatment induces a lasting higher proportion of anergic T cells (FIG. 15).

### Example 11

Ability of Bpep-NLC to deliver an intact Bpep.

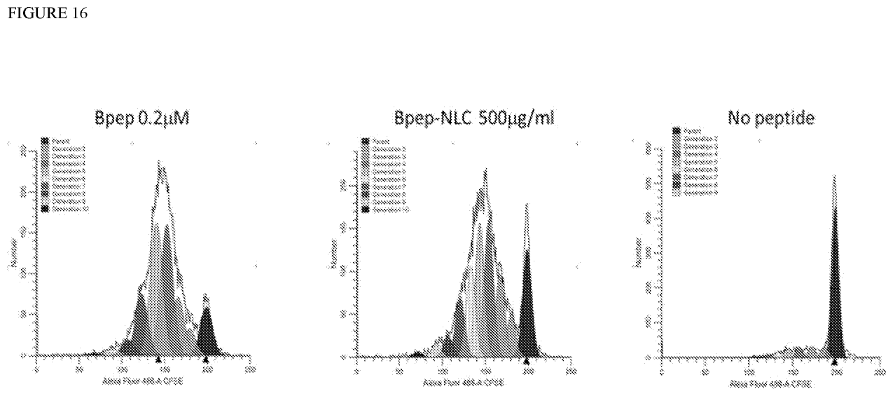

With the synthesis and further characterization of Bpep-NLc, we performed an experiment to determine if BMDC exposed to these particles would then be able to stimulate Bpep-reactive T cells (as proof of integrity of the Bpep in the LNp and of proper delivery to APCs). Mouse BMDC were exposed overnight to titrations of either soluble Bpep, Bpep-NLC, or control NLC, and then used to stimulate CFSE-stained T cells isolated from BDC12-4.1 TCR Tg mice (transgenic mice generating T cells specific for the insulin Bpep). Readout of this assay was the induction of T cell proliferation shown as progressive dilution of the intensity of CFSE staining. As shown in FIG. 16, Bpep-NLC were readily capable of providing an intact Bpep to BMDC that then induced significant proliferation of BDC12-4.1 T cells. Based on these encouraging results, we are now proceeding in the optimization of Bpep-NLC to increase their Bpep content and enable delivery at lower dosages.

### Example 12

HPLC determination of tofacitinib encapsulation efficiency and release.

To demonstrate synergism and efficacy of Bpep-LNp, and Tofa-LNp, it is important to characterize fully the loading capacity and kinetics of the drug delivery platform (NLC) to inform dosing schedules. Prior to characterization of the particles, high performance liquid chromatography (HPLC) methods were developed to measure accurately the tofacitinib (Tofa). Methods development included system suitability analysis and calibration. The mobile phase chosen for analysis was 1-octanesulfonic acid with 0.1% phosphoric acid (H₃PO₄) and acetonitrile (75:25, v/v). The analysis was performed isocratically with a flow rate of 0.25 mL/min and the detection wavelength was set to 290 nm. Each volume of accurately weighed Tofa standard sample was diluted to volume with diluent to obtain a standard curve (R²=1.000)

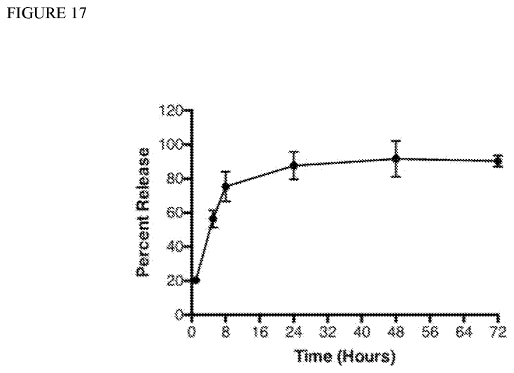

The release kinetic profile of Tofa was determined following dialysis of a 200 μL sample of each freshly prepared Tofa-NLC sample into a 5 ml volume of phosphate buffered saline (37° C., shaking) for a given time period. Three technical replicates of each sample were prepared and analyzed over a 72-hour time course. At specific time points, an aliquot of the buffer was collected for subsequent HPLC analysis. Our results indicate a biphasic release pattern, characterized by a burst in the first eight hours and then slower release up to approximately 30 hours. (FIG. 17 (12)). The system proved to be suitable for the analysis with a % RSD value of replicate injections of the 40 ug/mL standard solution of 0.41. Each accurately measured aliquot of the separated NLC solutions, containing Tofa, was diluted to volume with diluent and run on HPLC using the exact parameters as above. Tofa eluted at approximately 2.04 minutes for all samples and standards injected and the complete run time for all chromatographic runs was 6.0 minutes.

Determination of the Tofa-NLC drug loading, or encapsulation efficiency (EE %), was calculated following separation of the organic and aqueous phases using a spin-X UF concentrator (MWCO=5K) followed by centrifugation. Following this separation, the final volumes for both phases were analyzed using HPLC to quantify the amount of Tofa in each phase. Freshly prepared Tofa-NLCs were analyzed in parallel to quantify the total amount of Tofa in each starting preparation. Encapsulation efficiency was calculated by dividing the mass of Tofa in the organic phase by the total mass of Tofa and multiplying by 100. Three independent formulations were prepared and tested by this method, averaging a 79% EE (Table 2).

TABLE 2
Tofa-NLC Encapsulation Efficiency

| Formulation | Encapsulation Efficiency |
|---|---|
| 1 | 79 |
| 2 | 78 |
| 3 | 79 |
| Average | 79 +/− 1 |

The release kinetic profile of Tofa was determined following dialysis of a 200 μL sample of each freshly prepared Tofa-NLC sample into a 5 ml volume of phosphate buffered saline (37° C., shaking) for a given time period. Three technical replicates of each sample were prepared and analyzed over a 72-hour time course. At specific time points, an aliquot of the buffer was collected for subsequent HPLC analysis. Our results indicate a biphasic release pattern, characterized by a burst in the first eight hours and then slower release up to approximately 30 hours. Maximum release of Tofa under these experimental conditions was 90%. These methods will next be employed to assess peptide encapsulation and release kinetics from Bpep-NLC.

The hydrophobic ion pairing method was utilized prior to particle synthesis to increase peptide liposolubility. Implementation of this method involves complexation of the insulin Bpep (mimotope) with sodium deoxycholate, at a controlled pH. Complexation efficiency is determined through use of a BCA assay to quantitate the peptide remaining in the solution supernatant following mixing and incubation. Despite scaling up the pairing protocol by a factor of four, formulations maintained a pairing efficiency of approximately 85%, as assessed through BCA analysis of the pairing supernatant (Table 3). Our next steps involve determination of EE % and release kinetics using HPLC methods specifically developed for detection of the mimotope peptide.

TABLE 3
Bpep-NLC Formulations.

| | | | Solid to | | | |
|---|---|---|---|---|---|---|
| | | | Liquid | Lipid to | Processing | Cargo |
| Solid | Liquid | | Lipid | Surfactant | Temperature | Concentration |
| Lipid | Lipid | Surfactants | Ratio | Ratio | (° C.) | (mg/mL) |
| C19-C30 | Tocopherol | BrijO10 | 50:50- | 1:0.5-1:2 | 70-90 | 0.07-0.30 |
| | | Gelucire 44/14 | 90:10 | | | (Peptide) |
| | | Gelucire 48/16 | | | | 1.5-6.0 |
| | | Gelucire 50/13 | | | | (Immunosupressant) |

### Example 13

NLC-HLDI cellular uptake.

Our previously work of the incorporation of NLC were based on co-culture experiments with bone marrow derived dendritic cells. To better understand the impact that Tofa-NLC would have once injected in vivo, we performed additional experiments to assess ex vivo the incorporation of NLC by different cell types of splenic cells.

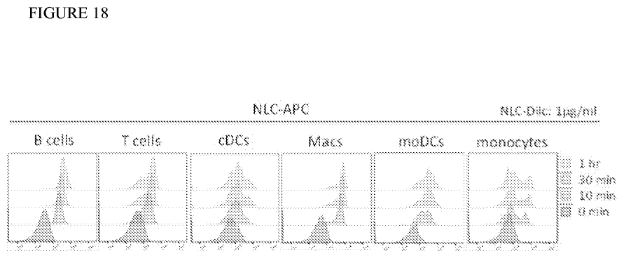

FIG. 18 shows that many subpopulations of splenocyte acquire NLC within 10 minutes and then continue, although slowly, to acquire more NLC. cDCs and monocytes show a modest incorporation of NLC, with the presence of a small subset characterized by very high uptake. B and T cells, as well as Macrophages and monocytic DC, all presented a significant uptake of NLCs.

### Example 14

NLC-HLDI in vivo biodistribution.

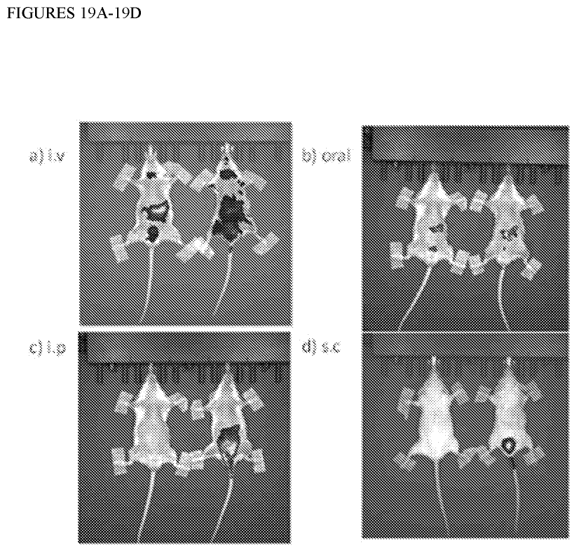

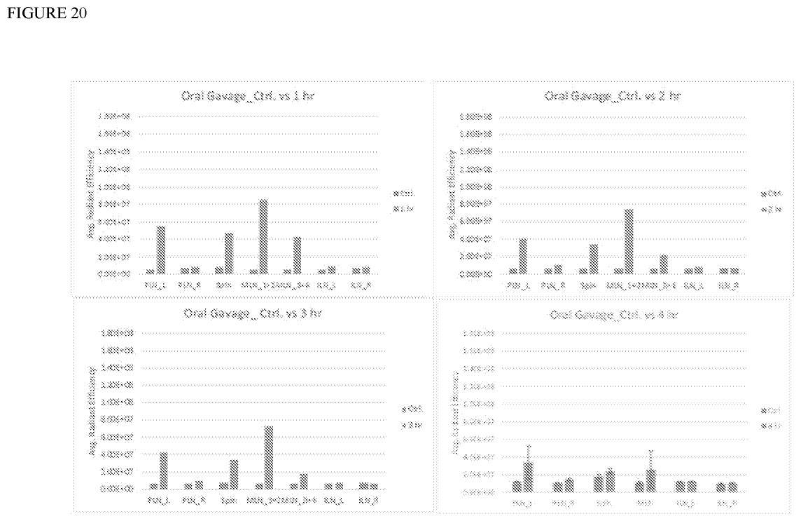

Several routes of administration have been tested to compare the biodistribution of NLC at various time points. Shown in FIG. 19 are representative images of live, whole animal fluorescence, taken via IVIS system at either 4 or 24 hours after NLC administration. We then focused on gavage administration and performed additional experiments to determine the peak time of particle incorporation and to study the in vivo cell tropism. Either 1, 2 or 3 hours after oral administration of 15 mg HLDI-NLC, spleen, pancreatic LNs, mesenteric LNs, and inguinal LNs were harvested and compared to no treatment controls. Via IVIS imaging and analysis, we determined that the signal peaks at 1 h, stabilizes at 2 and 3 h, then diminishes after 4 h (FIG. 20).

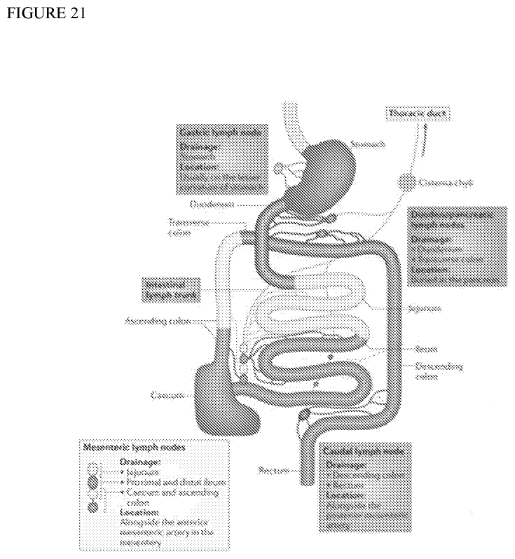

Intriguingly, this study revealed that only one of the two pancreatic lymph nodes, and only two of the four mesenteric lymph nodes, showed significant fluorescence signal after NLC treatment. Strikingly, this correlates with the different location and order of gastro-intestinal areas drained by each lymph node (FIG. 21). The left pancreatic LN and the first two mesenteric LNs drain the proximal section of the intestine whereas the right pancreatic LN and mesenteric LNs 3 and 4 drain the distal section of the intestine. We hypothesize that the particles have been digested or absorbed by the time they reach the distal section of the intestine, resulting in less fluorescent signal. Hence, we decided to base our future tests mainly on the left pancreatic LN and mesenteric LNs 1 and 2 EXAMPLE 15

The controlled generation of therapeutic-loaded particles requires an understanding of the dependencies of multiple processing parameters. We implemented the Design of Experiments (DOE) statistical method to understand the effects of these factors on the formation of Tofacitinib (Tofa)—loaded nanostructured lipid carriers (NLCs). The DOE of tofa-NLC processing parameters (i.e. solid lipid type, liquid lipid type, surfactant type, solid to liquid lipid ratio, lipid to surfactant ratio, tofa concentration, and processing temperature) were evaluated by completing 30 individual experimental runs. Formulation products were analyzed by assessing particle formation (yes/no), particle size, polydispersity, melting point, and latent heat of melting.

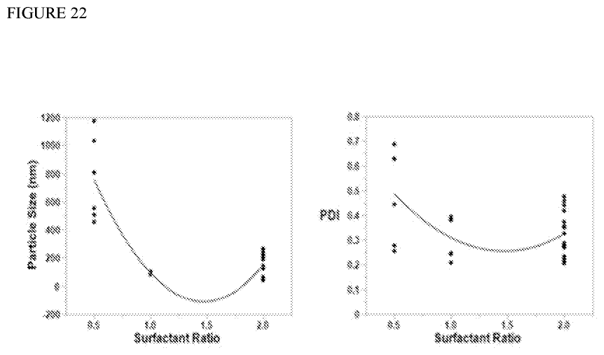

Our results indicated that lipid and surfactant type as well as lipid to surfactant ratio and processing temperature all impacted the formation of measurable particles, as assessed by dynamic light scattering (DLS). A statistical analysis determined which factors influenced the particle size, polydispersity index (PDI), melting point, and latent heat of melting of the lipid particles with more than 95% confidence. The analysis was also used to determine the pairs of parameters that are cross-correlated, if any. The particle size (P<0.0001) and PDI (P<0.0030) were solely impacted by the lipid to surfactant ratio, both resulting in a quadratic trend (FIG. 22). As expected, the main factor influencing the melting point was the composition of the solid and liquid lipids used. The crystallinity of the particles (as inferred from the latent heat of melting) was significantly influenced by the surfactant type, the lipid to surfactant ratio, the lipid blend, and the tofa concentration. A uniform nanoemulsion (determined by DLS) was maintained for only one day at maximal tofa loading, but this was demonstrated to be dose-dependent and stability improved with lower drug concentrations.

### Example 16

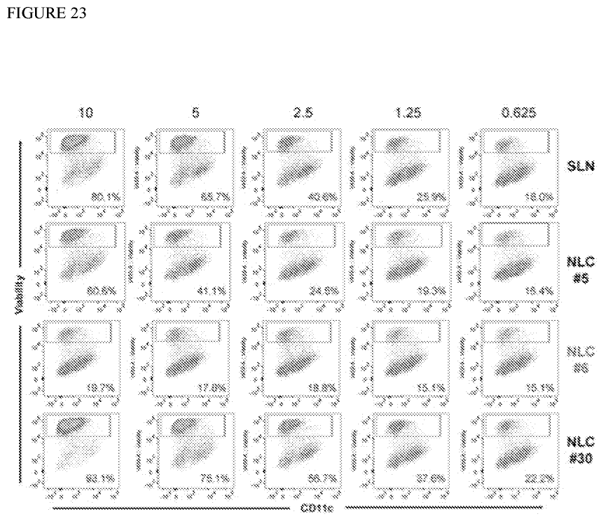

Having completed this first phase of optimization of Tofa-NLC formulation (focused on obtaining particles with size and properties that would maintain the desired unique properties of in vivo distribution and uptake of NLC), we performed a first screening of the toxicity of selected formulations on bone marrow-derived mouse dendritic cells. As indicated in FIG. 23, different formulations had very different effects on cell viability following a 24 hour incubation. Using this data, we identified a formulation, Tofa-NLC #6, with a very promising low toxicity profile and that will be subjected to additional cycles of optimization (i.e., toxicity and tofa release tunability) and implemented in both our in vitro and in vivo studies.

### Example 17

Shor term oral administration of Tofa-NLC reduces the maturation of antigen presenting cells (APC):

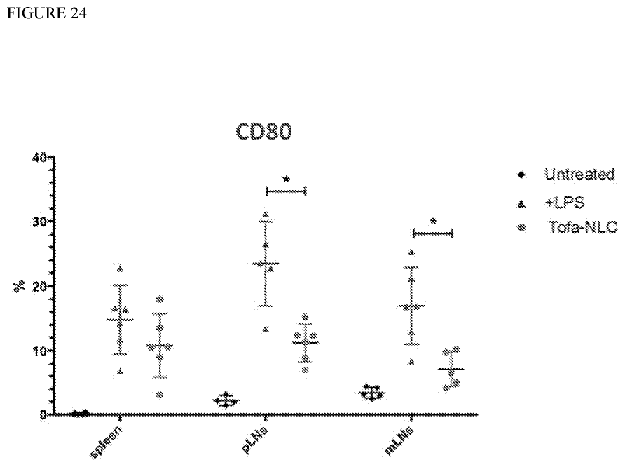

APC play an essential role in the activation and differentiation of diabetogenic effectors as well as in their regulation. Multiple reports suggest a connection between dysregulated activity of specific APC and diabetes development. Our results on the accumulation of anergic T cells suggest that Tofa-NLC could act by preventing the maturation of APC and favoring the conversion of diabetogenic T cells, interacting with immature APC, into anergic T cells. To test this hypothesis, we measured the response to an in vivo challenge with the bacterial product LPS. This maturative stimulus was administered between the 4th and 5th administration of Tofa-NLC and its effect quantified the day after the last LNp administration. Using a cytometric panel for maturation markers on different APC, we determined that Tofa-NLC administration reduced the response of certain APC subsets to danger signals (FIG. 24).

### Example 18

Approach for tuning therapeutic release by NLC.

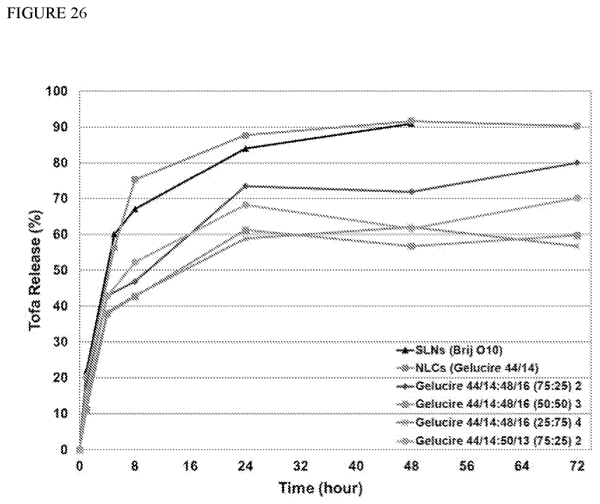

In order to modify the therapeutic release profile, we have explored the use of mixtures of mono, di, and triglycerides with PEG esters of fatty acids (commercial product family name is Gelucire). Gelucire containing only PEG esters are generally used in the preparation of fast/immediate/rapid release formulations, where Gelucire containing only glycerides or a mixture of glycerides and PEG esters are used in the preparation of sustained release formulations (Reference: doi.org/10.1016/j.fjps.2017.11.001). By mixing the surfactant chemistries having similar hydrophilic lipophilic balance (HBL), we were able to achieve tunability of the tofa release profile. We found that the release of Tofa from the modified NLC formulations was indeed altered by the surfactant composition and ratio. As the surfactant composition and ratio become more hydrophobic, the release of Tofa from the lipid core of the NP is slower (FIG. 26).

### Example 19

Approach for delivery of additional JAK inhibitors.

Our previous LNp formulations have been synthesized using the PIT method, which is amenable to inclusion of a wide variety of lipids, surfactants, and co-solvents, a feature that is exceptionally important for the encapsulation of cargo molecules with different chemistry. While the maximum drug loading in these vehicles is ultimately limited by the chemistry of the cargo molecule, the solubility can be modified somewhat through the addition of co-solvents in the lipid phase such as DMSO and ethanol to increase the hydrophilicity in the lipid phase. Although co-solvents will tend to decrease the melting point and crystallinity, their concentration can be varied over a considerable range without completely disrupting the phase inversion or suppressing the freezing phase transition. Ultimately, the cargo solubility is a critical aspect for the therapeutic applications, since low solubility could limit the therapeutic effects in vivo.

We have previously encapsulated immunosuppressant drugs and JAK inhibitors into our LNp platform using the PIT method (Table 4) and anticipate similar outcome in encapsulating additional JAK inhibitors (e.g. Filgotinib), with comparable chemical characteristics. In our preliminary studies, we have utilized the above method for producing Filgo-NLC and were able to achieve a stable nanoemulsion. Experiments are underway to maximize and assess encapsulation of Filgo in the particles and evaluate their efficacy in vitro.

TABLE 4
Comparison of Drug Substances for Incorporation into LNp.

| Drug | Molecular Weight | Molecular | Solubility in Dimethyl |
|---|---|---|---|
| Substance | (g/mol) | Structure | Sulfoxide (mg/mL) |
| Repamycin* | 914.17 | 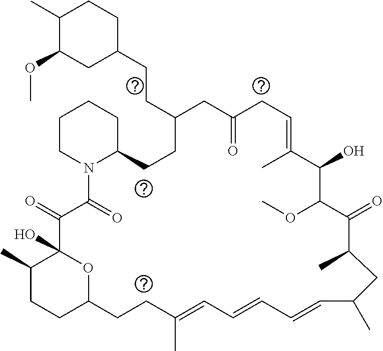 [SMILES: COC1C(=O)[C@H](C)CC(C)/C=C/C=C/C=C(\C)CCC2CC[C@@H](C)[C@@](O)(O2)C(=O)C(=O)N2CCCC[C@H]2CCC(CCC2CCC(C)[C@H](OC)C2)CC(=O)C/C=C(\C)[C@H]1O] | 200 |
| Tofacitinib* | 312.77 | 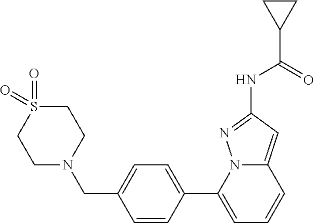 [SMILES: O=C(Nc1cc2cccc(-c3ccc(CN4CCS(=O)(=O)CC4)cc3)n2n1)C1CC1] | 100 |
| Filgotinib | 425.07 | 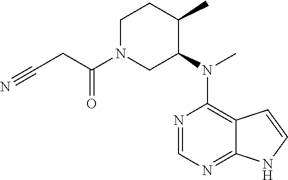 [SMILES: C[C@@H]1CCN(C(=O)CC#N)C[C@@H]1N(C)c1ncnc2[nH]ccc12] | 85 |

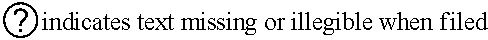

### Example 20

Impact of Bpep-LNp on the efficacy of Tofa-LNp to prevent T1D development in NOD mice.

It is thought that the impact of Tofa-LNp will be maximized by the co-administration of T1D autoantigen(s) and we expect a LNp formulation to provide the best result. The first test will identify the impact that the oral administration of Bpep-LNp alone (given using the same regimen adopted for Tofa-LNp) has on diabetes onset in NOD mice (3 week Vs 10 week old). We will then test the impact of the combination with our ECoB strategy, where Bpep-LNp will be administered together with Tofa-LNp. In all these experiments we will assess: diabetes onset, insulitis score, proportion of B peptide-reactive T cells present in the periphery and pancreas, as well as appearance and function of protective Treg.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Skyler, J. S. Prevention and reversal of type 1 diabetes—past challenges and future opportunities. Diabetes Care 38, 997-1007 (2015).
2. Tang, Q. et al. Central role of defective interleukin-2 production in the triggering of islet autoimmune destruction. Immunity 28, 687-97 (2008).
3. D′Alise, A. M. et al. The defect in T-cell regulation in NOD mice is an effect on the T-cell effectors. Proc Natl Acad Sci USA 105, 19857-62 (2008).
4. Monti, P. et al. Rapamycin monotherapy in patients with type 1 diabetes modifies CD4+CD25+FOXP3+regulatory T-cells. Diabetes 57, 2341-7 (2008).
5. Buckner, J. H. Mechanisms of impaired regulation by CD4(+)CD25(+)FOXP3(+) regulatory T cells in human autoimmune diseases. Nat Rev Immunol 10, 849-59 (2010).
6. D′Alise, A. M., Ergun, A., Hill, J. A., Mathis, D. & Benoist, C. A cluster of coregulated genes determines TGF-beta-induced regulatory T-cell (Treg) dysfunction in NOD mice. Proc Natl Acad Sci USA 108, 8737-42 (2011).
7. Bluestone, J. A., Tang, Q. & Sedwick, C. E. T regulatory cells in autoimmune diabetes: past challenges, future prospects. J Cin Immunol 28, 677-84 (2008).
8. Tarbell, K. V., Yamazaki, S., Olson, K., Toy, P. & Steinman, R. M. CD25+CD4+T cells, expanded with dendritic cells presenting a single autoantigenic peptide, suppress autoimmune diabetes. J Exp Med 199, 1467-77 (2004).
9. Tang, Q. et al. In vitro-expanded antigen-specific regulatory T cells suppress autoimmune diabetes. J Exp Med 199, 1455-65 (2004).
10. Masteller, E. L. et al. Expansion of functional endogenous antigen-specific CD4+CD25+regulatory T cells from nonobese diabetic mice. J Immunol 175, 3053-9 (2005).
11. Tarbell, K. V. et al. Dendritic cell-expanded, islet-specific CD4+CD25+CD62L+regulatory T cells restore normoglycemia in diabetic NOD mice. J Exp Med 204, 191-201 (2007).
12. Long, S. A., Buckner, J. H. & Greenbaum, C. J. IL-2 therapy in type 1 diabetes: “Trials” and tribulations. Clin Immunol 149, 324-31 (2013).
13. Reed, J. C. & Herold, K. C. Thinking bedside at the bench: the NOD mouse model of T1DM. Nat Rev Endocrinol 11, 308-14 (2015).
14. Roep, B. O., Atkinson, M. & von Herrath, M. Satisfaction (not) guaranteed: re-evaluating the use of animal models of type 1 diabetes. Nat Rev Immunol 4, 989-97 (2004).
15. Roep, B. O. & Tree, T. I. Immune modulation in humans: implications for type 1 diabetes mellitus. Nat Rev Endocrinol 10, 229-42 (2014).
16. Jenkins, M. K. The ups and downs of T cell costimulation. Immunity 1, 443-6 (1994).
17. Ford, M. L., Adams, A. B. & Pearson, T. C. Targeting co-stimulatory pathways: transplantation and autoimmunity. Nat Rev Nephrol 10, 14-24 (2014).
18. Turka, L. A. et al. T-cell activation by the CD28 ligand B7 is required for cardiac allograft rejection in vivo. Proc. Natl. Acad. Sci. USA 89, 11102-11105 (1992).
19. Pearson, T. C. et al. Analysis of the B7 costimulatory pathway in allograft rejection. Transplantation 63, 1463-69 (1997).
20. Pearson, T. C. et al. Transplantation tolerance induced by CTLA4-Ig. Transplantation 57, 1701-1706 (1994).
21. Sayegh, M. H. et al. CD28—B7 blockade after alloantigenic challenge in vivo inhibits Th1 cytokines but spares Th2. J Exp. Med. 181, 1869-74 (1995).
22. Lenschow, D. et al. Long-term survival of xenogeneic pancreatic islet grafts induced by CTLA4Ig. Science 257, 789-792 (1992).
23. von Herrath, M. & Nepom, G. T. Animal models of human type 1 diabetes. Nat Immunol 10, 129-32 (2009).
24. McKinney, E. F., Lee, J. C., Jayne, DR., Lyons, P. A. & Smith, K. G. T-cell exhaustion, co-stimulation and clinical outcome in autoimmunity and infection. Nature 523, 612-6 (2015).
25. Lenschow, D. J. et al. Differential effects of anti-B7-1 and anti-B7-2 monoclonal antibody treatment on the development of diabetes in the nonobese diabetic mouse. J Exp Med 181, 1145-55 (1995).
26. Larsen, C. P. et al. Long-term acceptance of skin and cardiac allografts after blocking CD40 and CD28 pathways. Nature 381, 434-438 (1996).
27. Ford, M. L., Stempora, L. L. & Larsen, C. P. CD28 blockade induces division-dependent downregulation of interleukin-2 receptor alpha. Transpl Immunol 24, 94-9 (2011).
28. Riella, L. V. & Sayegh, M. H. T-cell co-stimulatory blockade in transplantation: two steps forward one step back! Expert Opin Biol Ther 13, 1557-68 (2013).
29. Orban, T. et al. Costimulation modulation with abatacept in patients with recent-onset type 1 diabetes: follow-up 1 year after cessation of treatment. Diabetes Care 37, 1069-75 (2014).
30. Orban, T. et al. Co-stimulation modulation with abatacept in patients with recent-onset type 1 diabetes: a randomised, double-blind, placebo-controlled trial. Lancet 378, 412-9 (2011).
31. Chong, A. S. & Alegre, M. L. The impact of infection and tissue damage in solid-organ transplantation. Nat Rev Immunol 12, 459-71 (2012).
32. Lichtman, A. H., Chin, J., Schmidt, J. A. & Abbas, A. K. Role of interleukin 1 in the activation of T lymphocytes. Proc Natl Acad Sci USA 85, 9699-703 (1988).
33. Rochman, I., Paul, W. E. & Ben-Sasson, S. Z. IL-6 increases primed cell expansion and survival. J Immunol 174, 4761-7 (2005).
34. Ben-Sasson, S. Z. et al. IL-1 acts directly on CD4 T cells to enhance their antigen-driven expansion and differentiation. Proc Natl Acad Sci USA 106, 7119-24 (2009).
35. Rao, D. A. et al. Interleukin (IL)-1 promotes allogeneic T cell intimal infiltration and IL-17 production in a model of human artery rejection. J Exp Med 205, 3145-58 (2008).
36. Jones, S. A., Scheller, J. & Rose-John, S. Therapeutic strategies for the clinical blockade of IL-6/gpl30 signaling. J Clin Invest 121, 3375-83 (2011).
37. McKay, D., Shigeoka, A., Rubinstein, M., Surh, C. & Sprent, J. Simultaneous deletion of MyD88 and Trif delays major histocompatibility and minor antigen mismatch allograft rejection. Eur J Immunol 36, 1994-2002 (2006).
38. Thornley, T. B. et al. Type 1 IFN mediates cross-talk between innate and adaptive immunity that abrogates transplantation tolerance. J Immunol 179, 6620-9 (2007).
39. Walker, W. E. et al. Absence of innate MyD88 signaling promotes inducible allograft acceptance. J Immunol 177, 5307-16 (2006).
40. Shen, H. & Goldstein, D. R. IL-6 and TNF-alpha synergistically inhibit allograft acceptance. J Am Soc Nephrol 20, 1032-40 (2009).
41. Gronholm, J. & Lenardo, M. J. Novel diagnostic and therapeutic approaches for autoimmune diabetes—a prime time to treat insulitis as a disease. Clin Immunol 156, 109-18 (2015).
42. Pujol-Autonell, I. et al. Use of autoantigen-loaded phosphatidylserine-liposomes to arrest autoimmunity in type 1 diabetes. PLoS One 10, e0127057 (2015).
43. Harrison, L. C. et al. Antigen-based vaccination and prevention of type 1 diabetes. Curr Diab Rep 13, 616-23 (2013).
44. Wan, X. & Zaghouani, H. Antigen-specific therapy against type 1 diabetes: mechanisms and perspectives. Immunotherapy 6, 155-64 (2014).
45. Gibson, V. B. et al. Proinsulin multi-peptide immunotherapy induces antigen-specific regulatory T cells and limits autoimmunity in a humanized model. Clin Exp Immunol 182, 251-60 (2015).
46. Effects of insulin in relatives of patients with type 1 diabetes mellitus. New England Journal of Medicine 346, 1685-1691 (2002).
47. Zhang, Z. J., Davidson, L., Eisenbarth, G. & Weiner, H. L. Suppression of diabetes in nonobese diabetic mice by oral administration of porcine insulin. Proc Natl Acad Sci USA 88, 10252-6 (1991).
48. Harrison, L. C., Dempsey-Collier, M., Kramer, D. R. & Takahashi, K. Aerosol insulin induces regulatory CD8 gamma delta T cells that prevent murine insulin-dependent diabetes. J Exp Med 184, 2167-74 (1996).
49. Moroz, E., Matoori, S. & Leroux, J. C. Oral delivery of macromolecular drugs: Where we are after almost 100 years of attempts. Adv Drug Deliv Rev (2016).
50. Ansari, M. J. et al. Enhanced oral bioavailability of insulin-loaded solid lipid nanoparticles: pharmacokinetic bioavailability of insulin-loaded solid lipid nanoparticles in diabetic rats. Drug Deliv, 1-8 (2015).
51. Toorisaka, E. et al. An enteric-coated dry emulsion formulation for oral insulin delivery. J Control Release 107, 91-6 (2005).
52. Tirnaksiz F, A. S., Celebi N. Nanoemulsions as drug delivery systems.
Nanoemulsions as drug delivery systems. Colloids in Drug Delivery, 221-244 (2010).
53. Mei Z, C. H., Weng T, Yang Y, Yang X. Solid lipid nanoparticle and microemulsion for topical delivery of triptolide. European Journal of Pharmaceutics and Biopharmaceutics 56, 189-196 (2003).
54. Liu J, H., W, Chen, H, Ni Q, Huibi X, Yang X. Isotretinoin-loaded solid lipid nanoparticles with skin targeting for topical delivery. International Journal of Pharmaceutics 328, 191-195 (2007).
55. Danhier, F. et al. PLGA-based nanoparticles: an overview of biomedical applications. J Control Release 161, 505-22 (2012).
56. Wissing, S. A., Kayser, O. & Muller, R. H. Solid lipid nanoparticles for parenteral drug delivery. Adv Drug Deliv Rev 56, 1257-72 (2004).
57. Ali Khan, A., Mudassir, J., Mohtar, N. & Darwis, Y. Advanced drug delivery to the lymphatic system: lipid-based nanoformulations. Int J Nanomedicine 8, 2733-44 (2013).
58. Shete, H. & Patravale, V. Long chain lipid based tamoxifen NLC. Part I: preformulation studies, formulation development and physicochemical characterization. Int J Pharm 454, 573-83 (2013).
59. Shete, H., Chatterjee, S., De, A. & Patravale, V. Long chain lipid based tamoxifen NLC. Part II: pharmacokinetic, biodistribution and in vitro anticancer efficacy studies. Int J Pharm 454, 584-92 (2013).
60. Baek, J. S., So, J. W., Shin, S. C. & Cho, C. W. Solid lipid nanoparticles of paclitaxel strengthened by hydroxypropyl-beta-cyclodextrin as an oral delivery system. Int J Mol Med 30, 953-9 (2012).
61. Trevaskis, N. L., Kaminskas, L. M. & Porter, C. J. From sewer to saviour—targeting the lymphatic system to promote drug exposure and activity. Nat Rev Drug Discov 14, 781-803 (2015).
62. Delong, T. et al. Pathogenic CD4 T cells in type 1 diabetes recognize epitopes formed by peptide fusion. Science 351, 711-4 (2016).
63. O'Shea, J. J., Kontzias, A., Yamaoka, K., Tanaka, Y. & Laurence, A. Janus kinase inhibitors in autoimmune diseases. Ann Rheum Dis 72 Suppl 2, ii111-5 (2013).
64. Wojciechowski, D. & Vincenti, F. Tofacitinib in kidney transplantation. Expert Opin Investig Drugs 22, 1193-9 (2013).
65. Calderon-Colon, X. et al. Synthesis of sub-10 nm solid lipid nanoparticles for topical and biomarker detection applications. Journal of Nanoparticle Research 16 (2014).
66. Calderon-Colon, X., Raimondi, G, Benkoski, J., Patrone, J. Solid Lipid Nanoparticles (SLNs) for Intracellular Targeting Applications. Journal of Visualized Experiments 105 (2015).
67. Su, C. A., Iida, S., Abe, T. & Fairchild, R. L. Endogenous memory CD8 T cells directly mediate cardiac allograft rejection. Am J Transplant 14, 568-79 (2014).
68. Tadros T, I. P., Esquena J, Solans C. Formation and stability of nano-emulsions. Advances in Colloid and Interface Science 108-109, 303-318 (2004).
69. P. Sevcikova, P. V., V. Kasparkova, J. Krejci. Formation, characterization and stability of nanoemulsions prepared by phase inversion. MACMESE'11 Proceedings of the 13th WSEAS international conference on Mathematical and computational methods in science and engineering, 132-137 (2011).
70. A. Forgiarini, J. E., C. González, C. Solans. Studies of the relation between pahse behavior and emulsificasion methods with nanoemulsion formation. Progr Colloid Polym Sci 115, 36-39 (2000).
71. Shang, L., Nienhaus, K. & Nienhaus, G. U. Engineered nanoparticles interacting with cells: size matters. J Nanobiotechnology 12, 5 (2014).
72. Beddoes, C. M., Case, C. P. & Briscoe, W. H. Understanding nanoparticle cellular entry: A physicochemical perspective. Adv Colloid Interface Sci 218, 48-68 (2015).
73. Muller, R. H., Shegokar, R. & Keck, C. M. 20 years of lipid nanoparticles (SLN and NLC): present state of development and industrial applications. Curr Drug Discov Technol 8, 207-27 (2011).
74. Muchow, M., Maincent, P. & Muller, R. H. Lipid nanoparticles with a solid matrix (SLN, NLC, LDC) for oral drug delivery. Drug Dev Ind Pharm 34, 1394-405 (2008).
75. Scholer, N., Hahn, H., Muller, R. H. & Liesenfeld, O. Effect of lipid matrix and size of solid lipid nanoparticles (SLN) on the viability and cytokine production of macrophages. Int J Pharm 231, 167-76 (2002).
76. Kathe, N., Henriksen, B. & Chauhan, H. Physicochemical characterization techniques for solid lipid nanoparticles: principles and limitations. Drug Dev Ind Pharm 40, 1565-75 (2014).
77. X. Calderón-Colón, J. B., M. Patchan, H. Le, M. Theodore, J. Sample, J. Patrone. in TechConnect World Conference and Expo 1-4 (2011).
78. Maldonado, R. A. et al. Polymeric synthetic nanoparticles for the induction of antigen-specific immunological tolerance. Proc Natl Acad Sci USA 112, E156-65 (2015).
79. Getts, D. R. et al. Therapeutic inflammatory monocyte modulation using immune-modifying microparticles. Sci Transl Med 6, 219ra7 (2014).
80. Hunter, Z. et al. A biodegradable nanoparticle platform for the induction of antigen-specific immune tolerance for treatment of autoimmune disease. ACS Nano 8, 2148-60 (2014).
81. Yeste, A., Nadeau, M., Bums, E. J., Weiner, H. L. & Quintana, F. J. Nanoparticle-mediated codelivery of myelin antigen and a tolerogenic small molecule suppresses experimental autoimmune encephalomyelitis. Proc Natl Acad Sci USA 109, 11270-5 (2012).
82. Varamini, P. & Toth, I. Recent advances in oral delivery of peptide hormones. Expert Opin Drug Deliv, 1-16 (2016).
83. Fonte, P. et al. Chitosan-coated solid lipid nanoparticles for insulin delivery. Methods Enzymol 508, 295-314 (2012).
84. Zhang, Z. H., Zhang, Y. L., Zhou, J. P. & Lv, H. X. Solid lipid nanoparticles modified with stearic acid-octaarginine for oral administration of insulin. Int J Nanomedicine 7, 3333-9 (2012).
85. Sarmento, B., Martins, S., Ferreira, D. & Souto, E. B. Oral insulin delivery by means of solid lipid nanoparticles. Int J Nanomedicine 2, 743-9 (2007).
86. Card, J. W. & Magnuson, B. A. A review of the efficacy and safety of nanoparticle-based oral insulin delivery systems. Am J Physiol Gastrointest Liver Physiol 301, G956-67 (2011).
87. Forgiarini A, Esquena J, Gonzalez C & C, S. Formation and stability of nano-emulsions in mixed nonionic surfactant systems. In: Koutsoukos P (ed) Trends in colloid and interface science XV, 118. Progress in Colloid and Polymer Science. Springer Berlin Heidelberg., 184-189 (2001).
88. R., F. The Design of Experiments (Oxford, England, 1935).
89. Jasinski, J. M. et al. Transgenic insulin (B:9-23) T-cell receptor mice develop autoimmune diabetes dependent upon RAG genotype, H-2g7 homozygosity, and insulin 2 gene knockout. Diabetes 55, 1978-84 (2006).
90. Sarikonda, G. et al. BDC12-4.1 T-cell receptor transgenic insulin-specific CD4 T cells are resistant to in vitro differentiation into functional Foxp3+T regulatory cells. PLoS One 9, e112242 (2014).
91. McDevitt, H. Specific antigen vaccination to treat autoimmune disease. Proc Natl Acad Sci USA 101 Suppi 2, 14627-30 (2004).
92. Liu, H. et al. Structure-based programming of lymph-node targeting in molecular vaccines. Nature 507, 519-22 (2014).

1. A lipid nanoparticle composition comprising one or more small molecule inhibitors of an inflammatory cytokine.

2. The lipid nanoparticle composition of claim 1, wherein the one or more small molecule inhibitors is Janus kinase (JAK) inhibitor.

3. The lipid nanoparticle composition of claim 2, wherein the Janus kinase (JAK) inhibitor is Tofacitinib and/or Filgotinib.

4. The lipid nanoparticle composition of claim 1, further comprising at least one or more additional biologically active agents.

5. The lipid nanoparticle composition of claim 4, wherein the at least one or more additional biologically active agents are peptides of proteins recognized as targets of autoimmune responses.

6. The lipid nanoparticle composition of claim 5, wherein the peptides of proteins recognized as targets of autoimmune responses comprise an insulin peptide.

7. The lipid nanoparticle composition of claim 6, wherein the insulin peptide is a fragment or portion of the insulin peptide.

8. The lipid nanoparticle composition of claim 7, wherein the insulin peptide is a fragment of a B peptide of insulin.

9. The lipid nanoparticle composition of claim 8, wherein the insulin peptide fragment comprises amino acids 9-23 of the B peptide of insulin.

10. The lipid nanoparticle composition of claim 1, wherein the one or more small molecule inhibitors of an inflammatory cytokine are labeled with one or more detectable moieties.

11. A method for preventing or treating Type 1 Diabetes in a subject in need thereof, the method comprising administering the lipid nanoparticle composition of claim 1.

12. The lipid nanoparticle composition of claim 4, wherein the at least one or more biologically active agents are labeled with one or more detectable moieties.
